(19) United States
(12) Patent Application Publication
(10) Pub. No.: US 2026/0308533 A1
(43) Pub. Date: Oct. 8, 2026

(54) MICROPATTERN-ASSISTED GENERATION OF TISSUE

(71) Applicant: NEW YORK UNIVERSITY, New York, NY (US)

(72) Inventor: Edwin ROSADO-OLIVIERI, New York, NY (US)

(21) Appl. No.: 19/637,189

(22) Filed: Apr. 2, 2026

Related U.S. Application Data
(60) Provisional application No. 63/782,365, filed on Apr. 2, 2025.

(51) Int. Cl. C12N 5/071

(52) U.S. Cl. CPC C12N 5/0697; C12N 5/0679; C12N 2500/30; C12N 2501/117; C12N 2501/15; C12N 2501/155; C12N 2501/16; C12N 2501/415; C12N 2506/02; C12N 2506/45; C12N 2513/00; C12N 2533/30; C12N 2533/52; C12N 2535/10; C12N 2537/00

(57) ABSTRACT

The invention is drawn to an innovative approach that leverages stereolithography-based micropatterning to generate adhesive micro-colonies of human embryonic stem cells (hESCs) with defined size and geometry. These glass-supported micropatterned arrays provide precise spatial confinement, allowing for controlled self-organization of hESC-derived TE tissues.

This application claims priority from U.S. provisional patent application no. 63/782,365, filed on Apr. 2, 2025, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records but otherwise reserves any and all copyright rights.

### FIELD OF THE INVENTION

This invention is directed to micropattern-assisted generation of trachea-esophageal tissues.

### BACKGROUND OF THE INVENTION

Malformations of the trachea-esophageal (TE) tract are among the most prevalent congenital abnormalities, occurring in 1 in 3,000 live births in the United States. These conditions, which can manifest as esophageal atresia (EA) and tracheoesophageal fistula (TEF), arise from disruptions in TE development during early embryogenesis. However, despite their clinical significance, the precise mechanisms underlying these malformations remain poorly understood. In particular, how dysregulated cellular and molecular processes drive deviations in cell differentiation and morphogenesis at the onset of EA/TEF remains unclear. A major challenge in addressing this knowledge gap is the lack of physiologically relevant, scalable models that allow for quantitative phenotypic analysis of human TE development and disease.

### SUMMARY OF THE INVENTION

An aspect of the invention is directed to a method of producing a trachea and esophagus on a microchip. In embodiments, the method can comprise culturing pluripotent stem cells in a culture medium cocktail to differentiate cells on the microchip as trachea tissue or esophagus tissue. In embodiments, the microchip can comprise glass-supported micro-islands of defined size and shape to support cell patterning.

Aspects of the invention are further drawn to a method for producing trachea or esophagus organoids. In embodiments, the method can comprise seeding definitive endoderm cells onto rectangular microfabricated culture substrates comprising elongated confinement geometries, including rectangular and elliptical shapes, that spatially confines the cells. In embodiments, the method can comprise contacting the cells with a TGFβ inhibitor and a BMP inhibitor to obtain patterned foregut tissues. In embodiments, the method can comprise contacting the patterned foregut tissues with a culture medium cocktail. For example, the culture medium cocktail can comprise KGF, BMP4, retinoic acid and a WNT agonist for tracheal tissue determination or KGF, Noggin, retinoic acid and a WNT agonist for esophageal tissue determination. In embodiments, the method can comprise culturing the definitive endoderm, foregut and patterned foregut cells in complete serum-free medium to obtain tracheal or esophageal organoids.

In embodiments, the microfabricated culture substrates can comprise a microchip.

In embodiments, the microchip can comprise rectangular hydrogel-coated domains (100-300 μm width; ≥500 μm length) separated by parallel hydrogel confinement walls to form well-like physical constraints.

In embodiments, the microchip can comprise micropatterned hydrogel substrates fabricated by projection sterolithography-mediated photopolymerization of poly(ethylene glycol) diacrylate (PEGDA), Lithium phenyl-2,4,6-trimethylbenzoylphosphinate (LAP) as photo-initiator and tartrazine as photo-absorber to generate hydrogel microstructures on glass-bottom culture vessels. For example, the photopolymerization can use about 20% 6K-8 KDa poly(ethylene glycol) diacrylate (PEGDA), about 34 mM Lithium phenyl-2,4,6-trimethylbenzoylphosphinate (LAP) and about 2.25 mM tartrazine.

In embodiments, the parallel hydrogel confinement walls can have a thickness exceeding cell height.

In embodiments, the hydrogel is coated with laminin.

In embodiments, the definitive endoderm cells are derived from embryonic stem cells (ESCs) or induced pluripotent stem cells (iPSCs).

In embodiments, the stem cells can be differentiated to the definitive endoderm cells using RPMI 1640 medium supplemented with 3 μM CHIR99021 and 100 ng/ml Activin A on day 1, 100 ng/mL Activin A and 0.2% deactivated fetal bovine serum (dFBS) on day 2, and 100 ng/ml Activin A plus 2% dFBS on day 3.

In embodiments, the TGFβ inhibitor can comprise SB431542 and the BMP inhibitor can comprise Dorsomorphin. For example, the concentration of SB431542 can be about 10 μm and of Dorsomorphin can be about 2 μm.

In embodiments, the definitive endoderm cells can be contacted with the TGFβ inhibitor and the BMP inhibitor for at least 3 days.

In embodiments, the WNT agonist can comprise CHIR9902.

In embodiments, for the tracheal tissue determination, the culture medium cocktail can provide a final concentration of the KGF of about 50 ng/ml, of the BMP4 of about 10 ng/ml, of the retinoic acid of about 100 nm and of the CHIR9902 of about 3 μM.

In embodiments, for the esophageal tissue determination, the culture medium cocktail can provide a final concentration of the KGF of about 50 ng/mL, of the Noggin of between about 10-200 ng/mL, of the retinoic acid of about 100 nm and of the CHIR9902 of about 3 μM.

In embodiments, the patterned foregut cells is contacted with the culture medium cocktail for about 7 days.

In embodiments, the medium containing the cocktail is applied apically to cover the patterned foregut tissues, with daily medium changes.

In embodiments, the complete serum-free medium comprises a complete serum-free differentiation medium (SFDM). For example, the SFDM comprises Advanced DMEM/F12, 15 mM HEPES, 2 mM L-glutamine, 1×B27 supplement and 1×N2 supplement.

In embodiments, the definitive endoderm, foregut and patterned foregut cells are cultured on laminin-coated micropattern glass coverslips.

In embodiments, contacting the foregut cells with the culture medium cocktail for tracheal tissue determination results in formation of bud-like structures and expression of NKX2.1. In embodiments, culturing the foregut cells with the culture medium cocktail for esophageal tissue determination results in formation of elongated tubular epithelia and expression of SOX2.

In embodiments, contacting the foregut cells with the tracheal differentiation cocktail promotes bud morphogenesis, characterized by increased lumen height and vertical aspect ratio, defined as a higher height relative to the longitudinal axis of the organoid.

In embodiments, contacting the foregut cells with the esophageal differentiation cocktail induces elongated tubular morphogenesis, resulting in an increased longitudinal elongation of lumens and a higher longitudinal aspect ratio, reflecting greater length relative to width with continuous, single lumens across each micropattern.

In embodiments, the definitive endoderm cells have a genetic mutation in, or abnormal expression of, a gene related to development of tracheal or esophageal tissue. For example, the gene can comprise CHD7, EFTUD2, ERCC4, FANCA, FANCB, FANCC, FAND1, FANG, FBN2, FGFR2, FLNA, FOXF1, FREM2, GDF3, GDF6, GLI3, MDM2, MEOX2, MID1, MKKS, MYCN, NKX2-1, NOG, PLEC1, PTEN, RARA, RARB, RBM8A, RIPK4, RMRP, SEMA3E, SMARCD1, SOX2, SOX4, SPECC1L, TBX1, TCOF1, WBP11, YY1, ZIC3, FOXF1, TBX1, FGFR2, BMP4, BMPR1A/B, GLI3, NOG, PTEN, or a combination thereof.

Still further, aspects of the invention are directed to human tracheal or esophageal tissue or organoid derived from the methods described herein.

Aspects of the invention are further drawn to a cultured tracheal or esophageal organoid derived from stem cells. In embodiments, the tracheal organoids exhibit radial tissue anisotropy surrounding bud primordia, including an edge-enriched orientation bias (increased Edge-to-Center Ratio). In embodiments, the esophageal organoids lack this feature.

Other objects and advantages of this invention will become readily apparent from the ensuing description.

### BRIEF DESCRIPTION OF THE FIGURES

FIG. 1 shows an example of trachea-esophageal morphogenesis. The development of the trachea and esophagus begins with the formation of the tracheal primordium, which undergoes budding morphogenesis. Subsequently, tissue morphogenesis drives the separation of the trachea and esophagus into anatomically and functionally distinct structures, forming the foundation of the aerodigestive tract.

FIG. 2 shows example generation of human tracheal/lung buds on micropatterns. A) Differentiation protocol of tracheal lung buds. B-C) Micrographs of human lung buds with segregation of SOX2+ airway and SOX9+ alveolar cells. D-E) Diagram of tracheal buds on micropatterns in vitro (D) or developing lungs in vivo (E).

FIG. 3 shows example reconstitution of TE morphogenesis on microchips. A) Protocol for the generation of TE organoids. B) Gradients of signaling pathways that influence TE development. C-F) Tracheal (E) vs esophageal (F) fate patterning in response to BMP and Noggin, respectively, is highly reproducible across micropatterns. High magnification images for representative colonies. Scale bar: 50 μm.

FIG. 4 shows example TE region-specific tissue morphogenesis under confinement. A-D) Modulation of BMP signaling leads to fate patterning (A) and region-specific morphogenetic outcomes such as esophageal-like tube elongation or tracheal-like budding as observed from 3D tissue growth analysis (B) as a well as cross-section (C) and side views (D). E) Proportion of colonies with budding tracheal or tubular esophageal morphogenesis upon modulation of BMP. Scale: 50 μm.

FIG. 5 shows example morphometric analysis of tissue topology via machine learning image analysis. A-C) Topological maps of organoid tissue and segmented lumens. D-E) Quantification of organoid volume (D) and height (E). F-I) Analysis of segmented lumens and quantification of lumen height (F), longitudinal elongation (G), longitudinal aspect ratio (H), and vertical aspect ratio (I).

FIG. 6 shows example compartment-specific tissue anisotropy during human TE development. A) Schematic framework illustrating the approach used to identify and quantify tissue anisotropy patterns. B-C) Representative examples of areas exhibiting high coherence (B) or misalignment (C), along with their corresponding single-cell coherence. D-G) Coherence maps of tracheal and esophageal differentiation at day 4 reveal a distinct circular pattern of tissue anisotropy observed exclusively in tracheal organoids. High-magnification insets are shown in (E) and (G). H) Radial profiles of anisotropy plotted from the colony center to the edge for tracheal and esophageal micropatterns. I) Quantification of the area under the curve (AUC) from (H). J) Edge-to-center ratio of tissue anisotropy.

FIG. 7 shows example pulsatile self-organizing tissue anisotropy during organogenesis. A) hPSC line to track nuclei (H2B), actin filaments (LifeAct) and tight junctions (ZO1). B) Tissue anisotropy maps showing the emergence of a circular pattern of anisotropy during bud morphogenesis. C) Radial profile of coherency displaying a high degree of alignment at the edge of the colony, and its disruption upon treatment with Cytochalasin D (CytoD). D) SATA allows time-resolved recording of anisotropy at a single cell level, and identification of cells exhibiting synchronized anisotropy. E) Cluster of cells with synchronized anisotropy identified by SATA. Time series of individual cells in grey and trend line in green. F) Measurement of time intervals of anisotropy clock and reproducibility across biological replicates. Scale bar: 100 μm.

FIG. 8 shows example single cell RNA-sequencing data. A-B) Single cell RNA-sequencing UMAP profiles of tracheal and esophageal tissues at day 7 of differentiation and identification of replicates (A) and cell types (B) in these tissues. C-D) UMAP expression profiles of SOX2 (C) and NKX2.1 (D). E) UMAP profiles of cell cycle stage of cells including cells in S and G2/M phases of the cell cycle. F) Heatmap of cell type-level expression of validated markers of the esophagus, trachea, foregut and mesenchyme. G-H) Integration and mapping of in vitro-derived micropatterned trachea-esophageal tissues onto a fetal dataset of lung and esophagus at weeks 11-15 of human post-conception. I) Hierarchical clustering of in vitro and reference in vivo cell types based on gene expression correlation. J) Pearson's correlation analysis of gene expression of cell types in in vitro-derived and in vivo reference datasets. K) Heatmap of gene expression of 138 genes associated with EA/TEF in patients and their expression profile in esophageal, tracheal and mesenchymal tissue of in vitro-derived trachea-esophageal organoids. L) Identification of 25 EA/TEF-associated genes that display tissue-specific expression identified through differential gene expression analysis.

FIG. 9 shows an example of loss of Noggin resulting in defective esophageal development. A-C) Induction of TE fate in wild-type and Noggin−/− cells in response to high (A-B) or varying (C) levels of Noggin, and quantification of cell proportions. D) Defective esophageal tube morphogenesis in Noggin−/− cells.

FIG. 10 shows example developmental signaling during human lung bud development A) Gradients of signaling pathways that influence TE development. B-D) Dose-dependent effects of KGF and BMP4 on tracheal progenitor differentiation (B-C), and of Noggin on esophageal patterning (D). **p<0.01, ****p<0.0001.

FIG. 11 shows example KGF-dependent epithelial tracheal bud morphogenesis. A) Quantification of tracheal bud epithelial area in micropattern colonies at varying doses of KGF. B) Micropattern colonies containing NKX2.1+ epithelial buds at increasing doses of KGF. C) Low magnification images of epithelial buds containing NKX2.1+ tracheal progenitors grown on microchips at high doses of KGF (50 ng/ml). **p<0.01, ****p<0.0001.

FIG. 12 shows example BMP and FGF pathway activity during TE patterning. A-B) pERK (A-B) and pSMAD1 (C-D) signaling activities at day 1, 4 and 9 of human foregut differentiation on microchips. **p<0.01, ****p<0.0001; scale bar, 50 μm.

FIG. 13 shows example differential BMP activity during TE development. A-D) Immunostaining for phospho-SMAD1 (pSMAD1), a readout of BMP pathway activity, and quantification of pSMAD1⁺ cells at days 2, 4, and 7 during tracheal (A-B) and esophageal (C-D) differentiation. E-G) pSMAD1 staining at the colony edge on day 4 of tracheal (E) and esophageal (F) differentiation, and the corresponding radial intensity profile of pSMAD1 activity during tracheal differentiation (G). Scale bar: 100 μm.

FIG. 14 shows example high-throughput predictive phenotypic analysis of human TE development. A) Large image databanks of TE organoids with patterning and morphogenesis markers. B) Autoencoder analysis for data encoding. C) Input images and reconstructed images are provided. D) UMAP-based representation of TE differentiation in response to BMP and Noggin stimulation. E) Differentiation scores for TE lineages based on probabilities obtained from random forest classifier analysis.

FIG. 15 shows example unbiased phenotypic profiling of Noggin−/− TE mini-organs. A) UMAP of latent features of Noggin−/− and wild-type samples. B) Differentiation scores under varying concentrations of BMP4 and Noggin. C-D) Proportion of samples classified as trachea, esophagus or precursor under BMP4, Noggin or no stimulation.

FIG. 16 shows examples of using a random forest classifier to predict tissue morphogenesis phenotypes. A) Random forest classifier accurately predicts bud morphogenesis based on latent features across replicates. B) Classifier reveals a dose-dependent effect of FGF4 on bud morphogenesis. C) MorphoStage regressor quantifies developmental progression and resolves stage-specific differences across FGF4 doses.

FIG. 17 shows reconstitution of human trachea-esophageal development and disease at scale. A) The transformation of the endodermal precursor into complex, functional organs. B) In vitro derivation of cells and tissues from hPSCs. C) Bioengineering: targeted reconstitution of human mini-organs. D) Trachea-esophageal morphogenesis and disease. E) Reconstituting human lung development on microchips. F) Reconstitution of trachea-esophageal morphogenesis in vitro. G) Fate-directed morphogenesis of the trachea-esophageal tract. H) High-resolution live-cell dynamics of human TE organogenesis. I) Cell contractility drives bud morphogenesis. J) Tissue-scale synchronization during bud organogenesis. K) AI-enabled phenotypic profiling of TE development. L) Reconstitution and predictive modeling of tracheoesophageal anomalies. M) Key takeways.

### DETAILED DESCRIPTION OF THE INVENTION

Aspects of the invention are drawn to a new approach that leverages stereolithography-based micropatterning to generate adhesive micro-colonies of human embryonic stem cells (hESCs) with defined size and geometry. These glass-supported micropatterned arrays provide precise spatial confinement, allowing for controlled self-organization of hESC-derived TE tissues. By combining this platform with advanced microscopy, genetic and pharmacological perturbations, and artificial intelligence (AI)-driven phenotypic profiling, a high-throughput system to analyze cell fate specification and morphogenesis during TE development and EA/TEF pathogenesis has been developed.

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Human trachea-esophageal (TE) development remains an experimental black box due to the inaccessibility of human embryos for experimentation. A bottleneck in the development of diagnostics and therapeutic interventions for esophageal atresia/tracheoesophageal fistula (EA/TEF) is the lack of models that allow for high-throughput, quantitative phenotypic analysis of human TE development and disease.

To address this challenge, directed differentiation protocols have been developed to generate esophageal and tracheal cells from hPSCs in 2D monolayers or 3D organoids. While these approaches have focused on deriving pure populations of functional cells for disease modeling and clinical applications, their potential to elucidate the mechanisms guiding human TE development and malformations remains largely untapped. They also suffer from high intra-and inter-organoid variability with respect to tissue complexity and morphology, limiting their suitability for high-throughput studies of cell fate and morphogenesis.

To circumvent these issues, described herein is micropattern-assisted differentiation to reconstitute and deconstruct human TE development, leveraging the self-organizing capabilities of human pluripotent stem cells (hPSCs) on microchips. The described micropattern platform overcomes limitations of current organoid systems, offering several key advantages: (1) robust and standardized generation of human tissues that mimic the complexity of the human fetal trachea and esophagus, (2) scalability and compatibility with high-throughput quantitative modalities to analyze thousands of mini-organs simultaneously, and (3) the ability to recapitulate morphogenetic events of human TE development which cannot be faithfully reproduced with current organoid systems. This micropattern system thus allows for scalable, high-resolution quantitative analyses of TE development in human tissues, providing an unprecedented framework to dissect the fundamental mechanisms coordinating cell fate and morphogenesis in the human trachea and esophagus. Furthermore, it can elucidate how genetic and environmental imbalances disrupt these processes, contributing to the development of EA/TEFs.

Herein, the term “patterning” can refer to initially equivalent cells becoming spatially organized into distinct domains (e.g., tube vs. bud) in response to differentiation signals that direct their fate. The microchips described herein provide for cells to “self pattern.” Herein, “micropatterning” describes microchips that can provide for cells to self pattern.

This new and powerful interdisciplinary approach integrates expertise in stem cell and developmental biology, genetics, systems biology, and high-resolution gene expression analysis to provide a comprehensive understanding of these processes. Beyond unveiling the cellular and molecular landscape of human TE development, the approach can pave a transformative path for leveraging organoid systems to shed light on fundamental principles of human organogenesis. By precisely mapping developmental abnormalities underlying organ malformations, this can open new avenues for targeted therapeutic interventions and the advancement of diagnostic technologies.

Aspects of the invention are directed to methods for producing an organ (e.g., a trachea or an esophagus) on a microchip. The methods described herein allow for controlled differentiation of pluripotent stem cells into trachea-like or esophagus-like tissue structures under defined microenvironmental conditions.

In embodiments, a method is provided for producing a trachea or esophagus on a microchip. The method comprises culturing pluripotent stem cells on a microchip in the presence of a culture medium cocktail (e.g., dFBS) configured to direct differentiation of the pluripotent stem cells toward tracheal tissue or esophageal tissue lineages. The methods are in vitro methods.

As used herein, “pluripotent stem cells” can refer to embryonic stem cells (ESCs), induced pluripotent stem cells (iPSCs), or genetically modified pluripotent cells that can differentiate into endodermal and epithelial lineage derivatives relevant to tracheal or esophageal tissue formation. The pluripotent stem cells can be human stem cells or derived from other mammalian species.

The term “microchip” can refer to a microfabricated device or substrate comprising one or more microscale features configured to support, constrain, modulate, or analyze biological samples, tissues, cells, or organoids. In various embodiments, a microchip can include, without limitation, micropatterned surfaces, microfluidic channels, wells, chambers, barriers, reservoirs, or combinations thereof, having characteristic dimensions on the order of micrometers to millimeters. A microchip can be fabricated from any suitable material, including but not limited to glass, polymers, elastomers, silicon, hydrogels, composites, or multilayer assemblies, and can incorporate surface chemistries, extracellular matrix coatings, or functionalized regions designed to control cell adhesion, geometry, spatial organization, morphogen exposure, or mechanical constraints. In certain embodiments, the microchip is configured to define colony geometry, tissue boundary conditions, or spatial morphogen gradients, and can be operably coupled to imaging systems, fluid delivery systems, sensors, or computational analysis pipelines. The term “microchip” expressly includes lab-on-a-chip devices, organ-on-a-chip platforms, micropatterned culture substrates, and microfluidic devices, whether passive or actively actuated.

In embodiments, the microchip comprises a substrate supporting patterned micro-islands formed of a material suitable for cell attachment and growth. In certain embodiments, the substrate comprises glass, although other optically transparent or biologically compatible materials can also be used. The micro-islands are supported on the glass substrate and are fabricated with defined sizes, geometries, and spatial arrangements to provide controlled cell patterning and organization. In some embodiments, the substrate (e.g., glass) can be coated with another material. In some embodiments, the substrate can be coated with a hydrogel material. In some embodiments, the hydrogel can be coated with laminin, or an extracellular matrix protein that allows for cell attachment to the hydrogel.

The micro-islands can be configured to regulate cell adhesion, colony size, and spatial confinement of the pluripotent stem cells during culture. By defining the size and shape of individual micro-islands, the microchip allows for reproducible control over cell density, cell-cell interactions, and local microenvironmental conditions, which together promote directed differentiation toward tracheal tissue or esophageal tissue phenotypes.

The term “micro-island” can refer to a spatially confined, microscale region defined on or within a substrate or microchip that is configured to support, contain, or constrain one or more cells, cell aggregates, tissues, or organoids. A micro-island can be defined by geometric boundaries, material contrast, surface chemistry, topographical features, fluidic isolation, or combinations thereof, and has characteristic dimensions on the order of micrometers to millimeters.

In various embodiments, a micro-island comprises a cell-permissive area bounded by cell-restrictive regions that limit lateral expansion, migration, or interaction with adjacent regions. Such boundaries can be formed using micropatterned coatings, physical barriers, differences in surface adhesion properties, or selectively functionalized materials. The micro-island geometry can be selected to impose defined boundary conditions that influence tissue architecture, morphogen exposure, mechanical stress distribution, or morphogenetic symmetry.

In certain embodiments, a micro-island is configured to host a single organoid, colony, or tissue structure, while in other embodiments, a micro-island can support multiple interacting cellular domains or developmental compartments. Micro-islands can be arranged in arrays, lattices, or arbitrary spatial configurations across a microchip to allow for parallelized experimentation, comparative analysis, or high-throughput screening.

The term “micro-island” expressly includes, without limitation, adhesive islands, micropatterned culture domains, confined epithelial regions, and isolated tissue niches formed within two-dimensional or three-dimensional culture environments. Unless otherwise specified, a micro-island may not require physical separation by walls or trenches and can be defined purely by functional or chemical patterning.

In some embodiments, the micro-islands are coated or functionalized with extracellular matrix proteins, peptides, or biomimetic coatings that enhance attachment, survival, and differentiation of the pluripotent stem cells. Non-limiting examples of such coatings include laminin, collagen, fibronectin, vitronectin, or combinations thereof.

The pluripotent stem cells are cultured on the micro-islands in a culture medium cocktail comprising one or more growth factors, signaling molecules, small molecules, or biological agents effective to induce differentiation toward tracheal or esophageal lineages. The composition of the culture medium cocktail can be varied over time to sequentially promote lineage specification, patterning, and maturation of the developing tissue.

In certain embodiments, the culture medium cocktail is configured to promote differentiation through definitive endoderm and foregut progenitor stages prior to specification into tracheal tissue or esophageal tissue. The differentiation process can be conducted under static conditions, perfusion conditions, or microfluidic flow conditions within the microchip.

As differentiation proceeds, the cells organize into tissue-like structures on the micro-islands that exhibit morphological, molecular, and/or functional characteristics of tracheal tissue or esophageal tissue. Such characteristics can include epithelial organization, expression of lineage-specific markers, formation of lumen-like structures, or development of tissue-specific cellular architectures.

The use of glass-supported micro-islands of defined size and shape allows for precise spatial control of tissue formation and facilitates real-time imaging, analysis, and manipulation of the developing tracheal or esophageal tissue on the microchip. The system further allows for reproducible tissue generation suitable for disease modeling, drug screening, toxicity testing, developmental studies, or regenerative medicine research.

Aspects of the invention are further directed to methods for producing organoids (e.g., tracheal organoids or esophageal organoids) using spatially confined differentiation of definitive endoderm cells on engineered microfabricated culture substrates. The systems and methods described herein allow for reproducible control of tissue patterning, lineage determination, and morphogenesis through the combined use of geometric confinement, defined signaling pathway modulation, and serum-free culture conditions.

In one embodiment, definitive endoderm cells are seeded onto rectangular microfabricated culture substrates comprising elongated confinement geometries. Such geometries can include rectangular, elliptical, or substantially elongated shapes configured to spatially confine the cells along a longitudinal axis. Spatial confinement promotes controlled tissue organization, limits lateral spreading, and enhances reproducibility of pattern formation during differentiation.

In some embodiments, the microfabricated culture substrate is provided as a microchip. The microchip can comprise rectangular hydrogel-coated domains having a width in the range of about 50 μm to about 500 μm, such as about 75 μm to about 400 μm, about 100 μm to about 300 μm, or any sub-range thereof. The domains can further have a length in the range of about 200 μm to about 8,000 μm or greater, such as at least about 300 μm, at least about 500 μm, about 500 μm to about 1,500 μm, about 1,000 μm to about 2,500 μm, about 1,500 μm to about 3,000 μm, about 2,000 μm to about 3,500 μm, about 2,500 μm to about 4,000 μm, about 3,000 μm to about 4,500 μm, about 3,500 μm to about 5,000 μm, about 4,000 μm to about 5,500 μm, about 4,500 μm to about 6,000 μm, about 5,000 μm to about 6,500 μm, about 5,500 μm to about 7,000 μm, about 6,000 μm to about 7,500 μm, about 6,500 μm to about 8,000 μm, or greater, depending on the desired tissue morphology. For example, the microchip can comprise rectangular hydrogel-coated domains having widths of about 100-300 μm and lengths of at least about 500 μm. The domains can be separated by parallel hydrogel confinement walls configured to form well-like physical constraints for cell culture.

In embodiments, the organoid structures described herein can be extended to lengths greater than 6,000 μm using microfluidic platforms and/or bioreactor-based culture systems to allow for long-term differentiation and maturation.

The parallel confinement walls can have a thickness exceeding the height of the cultured cells, thereby preventing cell escape or cross-migration between adjacent domains and maintaining independent organoid formation within each patterned region.

In certain embodiments, the microchip comprises micropatterned hydrogel substrates fabricated by projection stereolithography-mediated photopolymerization. In one non-limiting example, poly(ethylene glycol) diacrylate (PEGDA) is used as the hydrogel matrix, lithium phenyl-2,4,6-trimethylbenzoylphosphinate (LAP) is used as a photoinitiator, and tartrazine is used as a photo-absorber to generate precisely defined hydrogel microstructures on glass-bottom culture vessels.

The term “hydrogel” can refer to a hydrophilic, three-dimensional polymeric network that can of retain a substantial amount of water or aqueous solution while maintaining structural integrity. Hydrogels can be formed from natural polymers, synthetic polymers, or combinations thereof, and can be crosslinked through physical interactions, chemical bonds, ionic interactions, photo-induced reactions, or combinations thereof.

In embodiments, a hydrogel is configured to support, encapsulate, embed, or interface with cells, tissues, organoids, or extracellular matrix components. The mechanical, chemical, and transport properties of the hydrogel—including but not limited to stiffness, porosity, degradability, permeability, and viscoelastic behavior—can be selected or tuned to modulate cellular behavior, tissue morphogenesis, differentiation, or response to morphogens.

In embodiments, a hydrogel can comprise or be functionalized with extracellular matrix proteins, peptides, growth factors, morphogens, adhesion ligands, or other bioactive components. The hydrogel can be spatially patterned, locally modified, or arranged in gradients to provide defined biochemical or mechanical cues. Hydrogels can be used as bulk matrices, surface coatings, confined regions (including within micro-islands), or structural elements of a microchip.

The term “hydrogel” expressly includes, without limitation, physically crosslinked gels, chemically crosslinked gels, interpenetrating polymer networks, composite hydrogels, and dynamically tunable or stimuli-responsive hydrogels.

The hydrogel domains and/or glass surfaces can be coated with extracellular matrix proteins to promote cell attachment and differentiation. In some embodiments, the hydrogel is coated with laminin. Additionally, the definitive endoderm, foregut, and patterned foregut cells can be cultured on laminin-coated micropatterned glass coverslips.

The definitive endoderm cells used in the disclosed methods can be derived from pluripotent stem cells, including embryonic stem cells (ESCs) or induced pluripotent stem cells (iPSCs). In some embodiments, definitive endoderm cells correspond to cells generated during embryonic gastrulation or cells exhibiting molecular and functional characteristics thereof. In some embodiments, the definitive endoderm cells are able to differentiate into epithelial lineages of the digestive and/or respiratory tracts, as well as other endoderm-derived organs. In certain embodiments, these cells can be further specified into foregut progenitor cells. In some embodiments, foregut cells are multipotent and can differentiate into esophageal and/or tracheal tissues, including structured epithelial organoids that recapitulate features of native foregut-derived tubular architectures.

In some embodiments, cellular differentiation of stem cells to definitive endoderm is induced using sequential administration of morphogens and serum components according to a multi-day protocol, wherein:
Day 1 Treatment: On a first day of differentiation, cells are exposed to a WNT pathway agonist and a transforming growth factor-β (TGF-β) pathway activator. In certain embodiments, the WNT pathway agonist comprises CHIR99021 at a concentration in the range of about 0.5 μM to about 10 μM, such as about 1 μM to about 5 μM, or about 2 μM to about 4 μM, and the TGF-B pathway activator comprises Activin A at a concentration in the range of about 25 ng/ml to about 300 ng/ml, such as about 50 ng/ml to about 200 ng/ml, or about 75 ng/ml to about 125 ng/mL. In one exemplary embodiment, CHIR99021 is provided at about 3 μM and Activin A is provided at about 100 ng/mL.
Day 2 Treatment: On a second day of differentiation, cells are cultured in the presence of Activin A and deactivated fetal bovine serum (dFBS). In certain embodiments, Activin A is present at a concentration in the range of about 25 ng/ml to about 300 ng/mL, such as about 50 ng/mL to about 200 ng/mL, or about 75 ng/ml to about 125 ng/ml, and dFBS is present at a concentration in the range of about 0.05% to about 1.0% (v/v), such as about 0.1% to about 0.5%, or about 0.15% to about 0.3%. In one exemplary embodiment, Activin A is provided at about 100 ng/ml and dFBS is provided at about 0.2%.
Day 3 Treatment: On a third day of differentiation, cells are exposed to Activin A and an increased concentration of dFBS. In various embodiments, Activin A is present at a concentration in the range of about 25 ng/mL to about 300 ng/mL, such as about 50 ng/mL to about 200 ng/mL, or about 75 ng/ml to about 125 ng/ml, and dFBS is present at a concentration in the range of about 0.5% to about 5% (v/v), such as about 1% to about 4%, or about 1.5% to about 2.5%. In one exemplary embodiment, Activin A is provided at about 100 ng/ml and dFBS is provided at about 2%.

In one exemplary protocol, pluripotent stem cells are differentiated into definitive endoderm cells using RPMI 1640 medium supplemented with:
1) about 3 μM CHIR99021 and about 100 ng/ml Activin A on day 1;
2) about 100 ng/ml Activin A and about 0.2% deactivated fetal bovine serum (dFBS) on day 2; and
3) about 100 ng/ml Activin A and about 2% dFBS on day 3.

These conditions promote efficient induction of definitive endoderm, in some embodiments, prior to seeding onto the microfabricated substrates.

In embodiments, 150-200K cells per well of an 8-well chamber slide are seeded. In embodiments, other carriers, such as 35 mm dishes, 4-well chamber slides and 96 multi-well plates, can be used. The number of cells seeded is optimized for each of the carriers.

Following seeding onto the elongated confinement geometries, the definitive endoderm cells are contacted with a transforming growth factor-β (TGF) inhibitor and a bone morphogenetic protein (BMP) inhibitor to generate patterned foregut tissues. In embodiments, the TGFβ inhibitor is SB431542 and the BMP inhibitor is Dorsomorphin.

In embodiments, the TGF-β receptor inhibitor comprises SB431542 at a concentration in the range of about 1 μM to about 30 μM, such as about 2 μM to about 20 μM, about 5 μM to about 15 μM, or about 8 μM to about 12 μM. In some embodiments, the BMP pathway inhibitor comprises Dorsomorphin at a concentration in the range of about 0.2 μM to about 10 μM, such as about 0.5 μM to about 5 μM, about 1 μM to about 3 μM, or about 1.5 μM to about 2.5 μM.

In certain embodiments, the cells are exposed to the inhibitors for a duration of at least about 24 hours, such as at least about 48 hours, at least about 72 hours, about 2 days to about 7 days, or longer, depending on the desired differentiation outcome. In one example, the cells are exposed to about 10 μM SB431542 and about 2 μM Dorsomorphin for at least about three days. This treatment induces foregut patterning within the spatially confined geometries.

The patterned foregut tissues are subsequently contacted with lineage-specific differentiation cocktails to direct development toward tracheal or esophageal organoids.

For tracheal tissue determination, the culture medium cocktail can comprise keratinocyte growth factor (KGF), BMP4, retinoic acid, and a WNT agonist. For esophageal tissue determination, the culture medium cocktail can comprise KGF, Noggin, retinoic acid, and a WNT agonist. In embodiments, the WNT agonist is CHIR99021.

In certain embodiments, a tracheal differentiation cocktail comprises a fibroblast growth factor, a bone morphogenetic protein, retinoic acid, and a WNT pathway agonist. In embodiments, Keratinocyte Growth Factor (KGF) is provided at a concentration in the range of about 10 ng/ml to about 200 ng/mL, such as about 25 ng/mL to about 100 ng/mL, about 40 ng/ml to about 75 ng/ml, or about 50 ng/mL. In embodiments, BMP4 is provided at a concentration in the range of about 1 ng/ml to about 50 ng/ml, such as about 5 ng/ml to about 25 ng/ml, about 8 ng/ml to about 15 ng/ml, or about 10 ng/mL. In embodiments, retinoic acid is provided at a concentration in the range of about 10 nM to about 1 μM, such as about 25 nM to about 500 nM, about 50 nM to about 200 nM, or about 100 nM. In embodiments, CHIR99021 is provided at a concentration in the range of about 0.5 μM to about 10 μM, such as about 1 μM to about 6 μM, about 2 μM to about 4 μM, or about 3 μM.

In one exemplary embodiment, the tracheal differentiation cocktail provides concentrations of about 50 ng/ml KGF, about 10 ng/ml BMP4, about 100 nM retinoic acid, and about 3 μM CHIR99021.

In embodiments, an esophageal differentiation cocktail comprises a fibroblast growth factor, a BMP pathway antagonist, retinoic acid, and a WNT pathway agonist. In embodiments KGF is provided at a concentration in the range of about 10 ng/ml to about 200 ng/ml, such as about 25 ng/ml to about 100 ng/mL, about 40 ng/mL to about 75 ng/ml, or about 50 ng/ml. In embodiments, Noggin is provided at a concentration in the range of about 5 ng/ml to about 500ng/mL, such as about 10 ng/mL to about 200 ng/mL, about 25 ng/mL to about 300 ng/mL, or about 50 ng/ml to about 150 ng/mL. In embodiments, retinoic acid is provided at a concentration in the range of about 10 nM to about 1 μM, such as about 25 nM to about 500 nM, about 50 nM to about 200 nM, or about 100 nM. In embodiments, CHIR99021 is provided at a concentration in the range of about 0.5 μM to about 10 μM, such as about 1 μM to about 6 μM, about 2 μM to about 4 μM, or about 3 μM.

In one exemplary embodiment, the esophageal differentiation cocktail provides concentrations of about 50 ng/mL KGF, about 10-200 ng/mL Noggin, about 100 nM retinoic acid, and about 3 μM CHIR99021.

The patterned foregut cells can be exposed to the differentiation cocktail for about seven days. In some embodiments, the differentiation medium is applied apically to cover the patterned tissues, with daily medium changes.

During differentiation, in some embodiments, the cells are maintained in complete serum-free medium. In certain embodiments, the medium comprises serum-free differentiation medium (SFDM). In embodiments, the SFDM comprises a basal medium supplemented with one or more buffering agents, amino acids, and defined nutrient supplements. In various embodiments, the basal medium comprises Advanced DMEM/F12 or a functionally equivalent basal medium. The basal medium can be supplemented. HEPES is provided at a concentration in the range of about 5 mM to about 50 mM, such as about 10 mM to about 30 mM, about 12 mM to about 25 mM, or about 15 mM. L-glutamine is provided at a concentration in the range of about 0.5 mM to about 6 mM, such as about 1 mM to about 4 mM, about 1.5 mM to about 3 mM, or about 2 mM. In some embodiments, L-glutamine can be replaced in whole or in part with a stabilized glutamine analog. B27 supplement is provided at a concentration in the range of about 0.25× to about 2×, such as about 0.5× to about 1.5×, about 0.75× to about 1.25×, or about 1×. N 2 supplement is provided at a concentration in the range of about 0.25× to about 2×, such as about 0.5× to about 1.5×, about 0.75× to about 1.25×, or about 1×.

In one example, the SFDM comprises Advanced DMEM/F12 supplemented with about 15 mM HEPES, about 2 mM L-glutamine, about 1×B27 supplement, and about 1×N2 supplement.

In certain embodiments, exposure to the tracheal differentiation cocktail results in formation of bud-like structures and expression of NKX2.1. Such tracheal organoids can exhibit bud morphogenesis characterized by increased lumen height and a higher vertical aspect ratio relative to the longitudinal axis of the organoid.

In embodiments, exposure to the esophageal differentiation cocktail results in the formation of elongated tubular epithelial structures and expression of SOX2. Esophageal organoids can display increased longitudinal elongation of lumens, resulting in a higher longitudinal aspect ratio with continuous, single lumens across each micropattern.

Tracheal organoids produced according to the disclosed methods can further exhibit radial tissue anisotropy surrounding bud primordia, including an edge-enriched orientation bias characterized by an increased Edge-to-Center Ratio.

In some embodiments, the definitive endoderm cells comprise a genetic mutation in, or abnormal expression of, one or more genes involved in tracheal or esophageal development. Non-limiting examples of genes include CHD7, EFTUD2, ERCC4, FANCA, FANCB, FANCC, FAND1, FANG, FBN2, FGFR2, FLNA, FOXF1, FREM2, GDF3, GDF6, GLI3, MDM2, MEOX2, MID1, MKKS, MYCN, NKX2-1, NOG, PLEC1, PTEN, RARA, RARB, RBM8A, RIPK4, RMRP, SEMA3E, SMARCD1, SOX2, SOX4, SPECC1L, TBX1, TCOF1, WBP11, YY1, ZIC3, FOXF1, TBX1, FGFR2, BMP4, BMPR1A/B, GLI3, NOG, PTEN, or a combination thereof.

Accordingly, the disclosed methods support in vitro modeling of congenital and acquired tracheoesophageal disorders.

The present disclosure further provides tracheal or esophageal tissues or organoids derived from the methods described herein. Such tissues can be human tissues derived from stem cells and can recapitulate key structural, molecular, and morphogenic features of native tracheal or esophageal tissue.

Aspects of the invention are further directed to methods for determining and classifying tissue morphogenesis of tracheal and esophageal organoids.

In one embodiment, a method for determining tissue morphogenesis comprises preparing a standardized mini-organ database comprising markers associated with trachea-esophageal patterning and morphogenesis. The database can include morphological, spatial, and molecular features derived from organoids produced using the disclosed microfabricated platforms.

The standardized mini-organ database is provided as input to an unsupervised autoencoder neural network. The autoencoder projects the database into a latent feature space that encodes patterns of tissue morphology and differentiation state.

Uniform manifold approximation and projection (UMAP) is then applied to reduce the latent space into two dimensions, generating a two-dimensional phenotypic map. The resulting two-dimensional data can reveal dose-dependent phenotypic responses to bone morphogenetic protein and Noggin signaling.

A random forest classifier is trained using the two-dimensional data and is subsequently used to classify organoids as tracheal, esophageal, or intermediate phenotypes.

In certain embodiments, the random forest classifier can further be used to predict tissue morphogenesis in time and space in response to external modulation, including changes in signaling pathway activation, growth factor concentrations, or environmental conditions.

In embodiments, the disclosed methods allow for prediction of tissue morphogenesis in response to external modulation by integrating time-resolved imaging, feature encoding, and machine learning-based modeling.

In certain embodiments, the method comprises generating time-resolved image datasets of micropatterned organoids cultured under defined morphogen conditions. Images can be acquired at a plurality of timepoints during differentiation, development, or maturation, and can include spatially resolved information characterizing tissue architecture, lumen geometry, epithelial organization, and cellular arrangement. Image acquisition can be performed using any suitable microscopy modality, including but not limited to confocal microscopy, fluorescence microscopy, phase-contrast microscopy, or light-sheet microscopy.

In some embodiments, the acquired image datasets are processed using a trained deep convolutional autoencoder. The autoencoder is configured to encode high-dimensional imaging data into a reduced-dimensional latent feature space while preserving relevant spatial relationships. Such relationships can include, without limitation, inter-epithelial spatial arrangement, tissue anisotropy, lumen positioning, and morphogenetic landmarks. The resulting latent feature representations serve as compact descriptors of morphogenetic state over time.

In embodiments, quantitative morphometric parameters are extracted from one or both of the latent feature representations and reconstructed images are generated by the autoencoder. Exemplary morphometric parameters include, but are not limited to, lumen aspect ratio, tissue anisotropy metrics, edge-to-center orientation bias, colony elongation index, bud height, bud curvature, lumen continuity, and branching topology. One or more additional geometric, topological, or texture-based features can also be derived, depending on the application.

In embodiments, the latent feature vectors and/or extracted morphometric parameters are computationally associated with experimental metadata. Such metadata can include, without limitation, morphogen identity, morphogen concentration, duration of morphogen exposure, micropattern or substrate geometry, and temporal stage of differentiation. This association can be performed on a per-sample, per-timepoint, or per-organoid basis.

In some embodiments, a machine learning model is trained using the extracted features and associated metadata. The model can comprise, for example, a random forest classifier, a random forest regressor, or a combination thereof. The trained model can be configured to classify morphogenetic phenotypes, predict developmental stage progression, and/or model dose-dependent morphogenetic trajectories across morphogen conditions.

In additional embodiments, the trained model is applied to previously unseen datasets to generate predictive outputs. Such predictions can include future morphogenetic outcomes at later timepoints, spatial patterning changes within developing colonies, and lineage bias or fate specification induced by genetic, epigenetic, or pharmacological perturbations.

In embodiments, the method further comprises generating a continuous morphogenesis prediction metric. This metric provides a quantitative representation of tissue development dynamics across time, morphogen conditions, or experimental perturbations, thereby enabling comparative analysis of morphogenetic trajectories.

In some embodiments, prediction accuracy and assay robustness are evaluated using one or more statistical performance criteria. By way of non-limiting example, accuracy can be validated by observing a coefficient of variation of less than 10 percent across biological replicates, a Z′ factor greater than or equal to 0.5, and/or concordance between predicted morphogenetic states and experimentally observed morphogenetic outcomes.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Reconstitution of Human Trachea-Esophageal Development and Congenital Malformations on Microchips, and Artificial Intelligence-Enabled Profiling of this Process

Aspects of the invention are drawn towards pluripotent stem cell (hPSC)-derived human mini-organs that resemble the developing trachea and esophagus on microchips. As described herein, micropattern-assisted differentiation is utilized for the in vitro generation of human embryonic tissues, which are glass-supported micro-islands of defined size and geometry. We have developed protocols to generate hPSC-derived tracheal and esophageal tissues with high reproducibility and robustness. These tissues undergo development in a way that mirrors in vivo development, with the formation of tracheal buds and esophageal tubes from the same precursor tissue. Moreover, we have deployed this model to study how pathogenic insults drive malformations of these tissues, in the form of esophageal atresia and tracheaesophageal fistula. To perform unbiased and predictive phenotypic profiling at a high-throughput scale, we have developed tools to generate large-scale image databanks of tracheaesophageal mini-organs on microchips and coupled that artificial intelligence-enabled analysis of trachea-esophageal developmental and malformations of these tissues. These are based on autoencoder neural network analysis and random forest tree classifier analysis. This pipeline allows us to accurately predict the effect of differentiation factors on trachea-esophageal development as well as the effects of pathogenic insults of this process at a high-throughput scale.

Human TE development remains an experimental black box due to the inaccessibility of human embryos for experimentation. A key bottleneck in the development of diagnostics and therapeutic interventions for congenital malformations is the lack of models that allow for high-throughput, quantitative phenotypic analysis of human tracheaesophageal development and disease. To address this challenge, directed differentiation protocols have been developed to generate esophageal and tracheal cells from hPSCs in 2D monolayers or 3D organoids. While these approaches have focused on deriving pure populations of functional cells for disease modeling and clinical applications, their potential to elucidate the mechanisms guiding human development and malformations remains largely untapped. They also suffer from high intra-and inter-organoid variability, limiting their suitability for high-throughput studies. To circumvent these issues, we have pioneered micropattern-assisted differentiation to reconstitute and deconstruct human TE development. The micropattern platform we have developed overcomes limitations of current organoid systems, offering key advantages: (1) robust and standardized generation of human tissues that mimic the human fetal trachea and esophagus, (2) scalability and compatibility with AI-enabled high-throughput modalities to analyze thousands of mini-organs simultaneously, and (3) the ability to recapitulate morphogenetic events which cannot be faithfully reproduced with current organoid systems. Our innovative micropattern system thus allows for scalable, high-resolution quantitative analyses of TE development in human tissues, providing an unprecedented framework to dissect the fundamental mechanisms coordinating cell fate and morphogenesis in the human trachea and esophagus. Furthermore, it will elucidate how genetic and environmental imbalances disrupt these processes, contributing to the development of EA/TEFs. Our unique and powerful interdisciplinary approach integrates expertise in stem cell and developmental biology, genetics, systems biology, and high-resolution gene expression analysis to provide a comprehensive understanding of these processes.

Directed differentiation protocols have been developed to generate esophageal and tracheal cells from hPSCs in 2D monolayers or 3D organoids. While these approaches have focused on deriving pure populations of functional cells for disease modeling and clinical applications, their potential to elucidate the mechanisms guiding human development and malformations remain largely untapped.

The combined model of mini-organs on microchips and high-throughput quantitative analysis has been validated for marker expression and performance metrics for artificial intelligence-enabled predictive classification. We have also validated these models in the context of disease-associated pathogenic insults, that allow us to predict disease outcomes in a way that mirrors what has been observed in patients and animal models.

Also provided herein is a high-throughput derivation of human mini-organs of the tracheaesophageal tract. We are interrogating how developmental signals in the form of recombinant proteins and small molecules have an effect on in vitro tissue development and morphogenesis. We are also combining this approach with CRISPR-based genetic screening of disease-associated genes to interrogate how pathogenic gene mutations drive defects in this process, as well as to delineate generalizable quantitative models of the disease. Embodiments are also drawn towards tracheaesophageal mini-organs from patient-derived samples, such as from blood, skin or amniotic fluid samples collected from patients with congenital malformations of the tracheaesophageal tract. Such embodiments can be used to delineate defects using our AI-phenotypic pipeline.

References cited in this example:
Jacob, A., & et al. (2017). Differentiation of Human Pluripotent Stem Cells into Functional Lung Alveolar Epithelial Cells. Cell Stem Cell, 21, 472-488.e10. PMID: 28965766
McCauley, K. B., & et al. (2017). Efficient Derivation of Functional Human Airway Epithelium from Pluripotent Stem Cells via Temporal Regulation of Wnt Signaling. Cell Stem Cell, 20 (6):844-857.e6. PMID: 28366587
Dye, B. R., & et al. (2015). In vitro generation of human pluripotent stem cell derived lung organoids. eLife, 4:e05098. PMID: 25803487
Chen, Y.-W., & et al. (2017). A three-dimensional model of human lung development and disease from pluripotent stem cells. Nature Cell Biology, 19, 542-549. PMID: 28436965
Huang, S. X., & et al. (2014). Efficient generation of lung and airway epithelial cells from human pluripotent stem cells. Nature Biotechnology, 32, 84-91. PMID: 24291815
A. J. Miller, B. R. Dye, D. Ferrer-Torres, D. R. Hill, A. W. Overeem, L. D. Shea, J. R. Spence. Generation of lung organoids from human pluripotent stem cells in vitro. Nat. Protoc., 14 (2019), pp. 518-540
Shacham-Silverberg, V., Wells, J. M., 2020. Generation of esophageal organoids and organotypic raft cultures from human pluripotent stem cells. Methods Cell Biol. 159, 1-22.
Trisno, S. L., Philo, K. E. D., McCracken, K. W., Cata, E. M., Ruiz-Torres, S., Rankin, S. A., Han, L., Nasr, T., Chaturvedi, P., Rothenberg, M. E., Mandegar, M. A., Wells, S. I., Zorn, A. M., Wells, J. M., 2018. Esophageal organoids from human pluripotent stem cells delineate Sox2 functions during esophageal specification. Cell Stem Cell 23, 501-515.
Wang, R., McCauley, K. B., Kotton, D. N., Hawkins, F., 2020. Differentiation of Human Airway-Organoids from Induced Pluripotent Stem Cells (iPSCs), 1 Ed. Elsevier Inc.

### Example 2—Reconstitution of Human Trachea-Esophageal Development and Disease at Scale

Combinatorial modulation of BMP, WNT, and FGF signaling directs hESCs to self-organize into tracheal or esophageal tissues with high reproducibility. hPSC-derived precursor cells exhibited region-specific morphogenetic behaviors on rectangular micropatterns: esophageal cells form elongated tubular structures, while tracheal cells undergo bud-like morphogenesis, mirroring their morphogenesis in vivo. Over seven days, esophageal tubes maintain their tubular architecture, while tracheal cells continue to develop into an extended budding structure, mirroring in vivo counterparts. To further validate the role of EA/TEF-associated genetic disruptions in TE development, CRISPR-mediated knockout of Noggin, a key BMP signaling regulator whose dysregulation has been implicated in EA/TEF, was used. Strikingly, Noggin−/− cells exhibited incomplete esophageal patterning and aberrant tracheal differentiation, a hallmark observed in EA/TEF patients. To systematically assess how EA/TEF-associated genetic mutations alter TE development, AI-based phenotypic analysis was implemented using an autoencoder neural network and classifier models applied to large-scale imaging databanks of mini-organs. Unbiased analysis confirmed that Noggin−/− cells exhibit an immature phenotype with (1) a low esophageal differentiation score, (2) precursor-like characteristics, and (3) a bias toward tracheal-like fate when deprived of patterning cues inducing TE patterning and region-specific morphogenesis.

This study highlights the self-organizing capabilities of hESCs when confined within micropatterned geometries and exposed to patterning signals that direct tracheal and esophageal fate specification, followed by region-specific morphogenesis. Our findings indicate that EA/TEF-associated genetic insults shift cellular competence and disrupt the balance between tracheal and esophageal differentiation. By leveraging our scalable micropatterning platform and AI-driven quantitative framework, this work establishes a powerful quantitative system to dissect human TE development and its perturbations in EA/TEF. Without wishing to be bound by theory, this framework can identify therapeutic targets and guide the development of new regenerative or pharmacological interventions to correct defective TE development in patients with EA/TEF.

### Example 3—Deconstructing Human TE Morphogenesis on Microchips

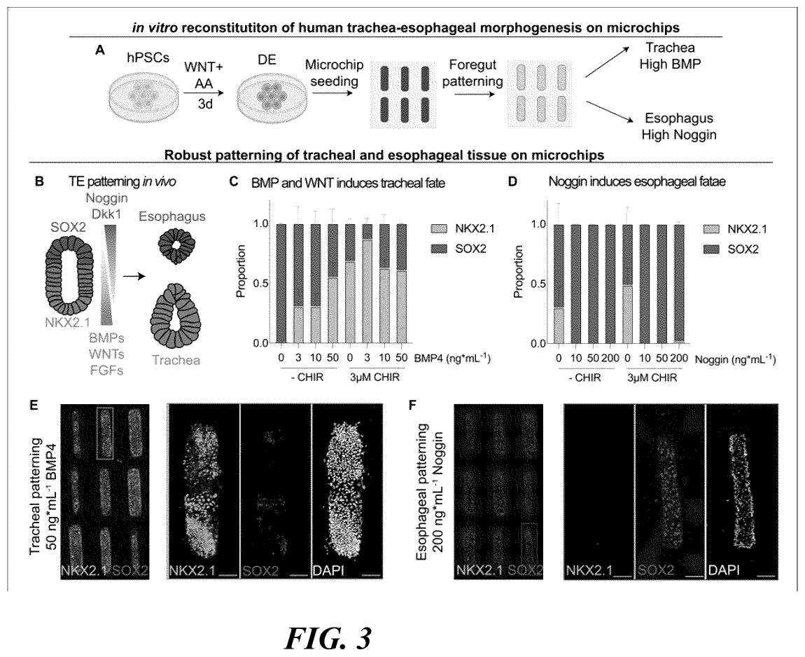

Leveraging the self-organizing capabilities of hPSC-derived foregut cells, a microchip system was established to guide their differentiation into tracheal and esophageal tissues (FIG. 3A). To replicate the natural geometry of the foregut and facilitate regionalized tissue patterning, we designed microchips featuring rectangular architectures (FIG. 3A). Prior to organogenesis, the tracheaesophageal tract emerges from the foregut, when it becomes regionally patterned along the dorso-ventral (D-V) axis by a core group of signaling pathways, including BMPs, WNTs, and FGFs (FIG. 3B). Given the pivotal role of BMP signaling in regulating tracheal versus esophageal fate—where its activation promotes tracheal identity and inhibition drives esophageal patterning—foregut cells were treated with high concentrations of the BMP pathway activator BMP4 or the inhibitor Noggin (FIG. 3A). We found that human tracheal differentiation relies on the combined modulation of WNT (CHIR99021), BMP4, and KGF (FGF7) signaling, whereas BMP inhibition by Noggin induces esophageal fate (FIG. 3C-F). Both lineages emerge with high efficiency across micropatterned foregut colonies within 4 days of stimulation (FIG. 3C-F). This approach allows for highly efficient and reproducible patterning of hPSC-derived foregut cells into NKX2.1+ tracheal or SOX2+ esophageal tissues across many micropatterned colonies and experimental replicates (FIG. 3C-F). This platform provides reproducible control over TE fate within defined geometries that mimic the architecture of the developing foregut prior to compartmental morphogenesis.

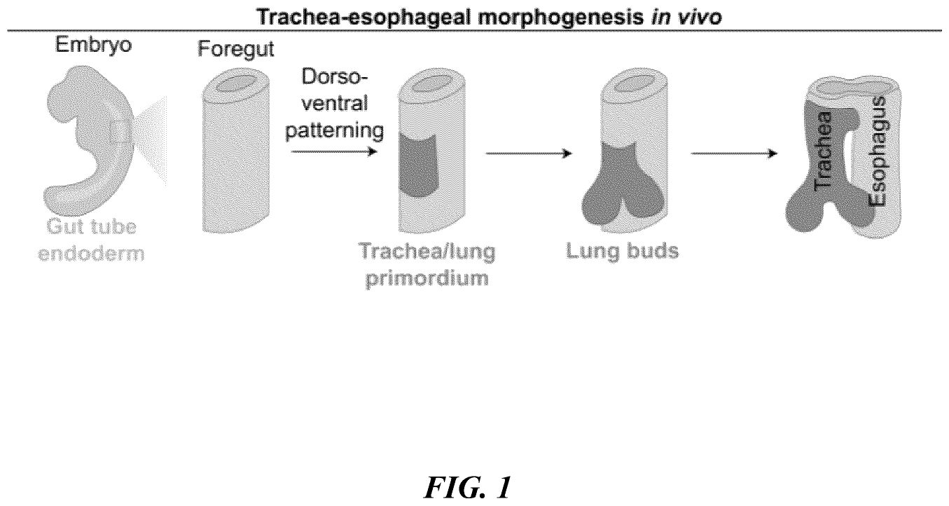

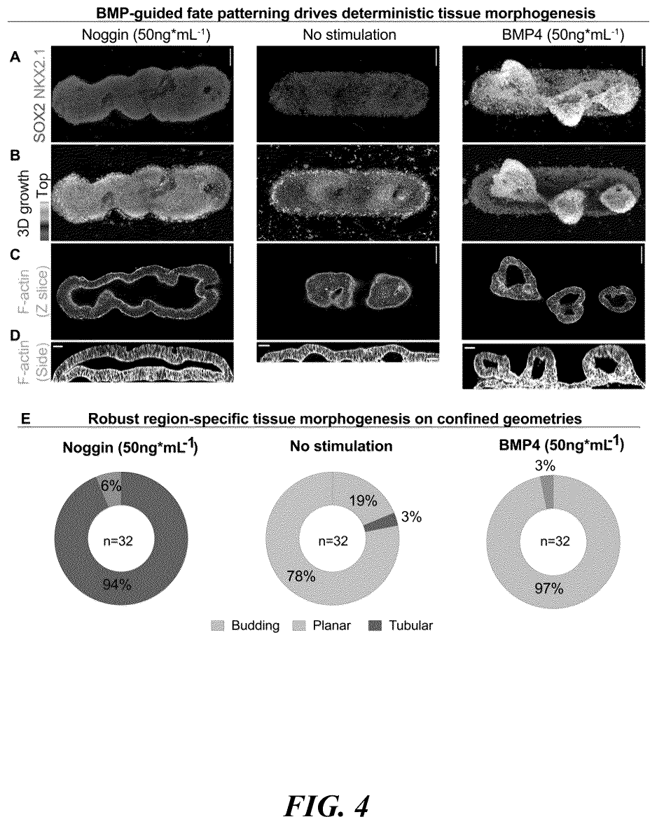

A critical morphogenetic determinant in TE development is the distinct behavior of tracheal and esophageal tissues: tracheal tissue undergoes bud morphogenesis, while esophageal tissue exhibits tube morphogenesis aligned along the rectangular axis of the foregut (FIG. 1). Disruptions in these region-specific morphogenetic processes can lead to incomplete development of the esophageal tube, as seen in EA, or a failure of the trachea to separate from the esophagus, as observed in TEF. hPSC-derived foregut cells exhibited region-specific morphogenetic behaviors: Noggin-induced SOX2⁺ esophageal cells formed elongated tubular structures, while BMP4-induced NKX2.1⁺ tracheal cells underwent bud-like morphogenesis (FIG. 4A-D), mirroring their morphogenesis in vivo (FIG. 1). Over 94% of TE organoids mirroring their morphogenesis in vivo (FIG. 4E), revealing robust and highly reproducible self-organization of TE progenitors into tracheal esophageal tissues resembling in vivo. Esophageal tubes retained their tubular architecture over 7 days, whereas tracheal cells continued to develop into an extended budding structure (FIG. 4A-D). To distinguish between tube elongation and bud formation, the eccentricity of the epithelium was quantified. Esophageal tissue exhibited an average eccentricity of 0.97, consistent with tubular elongation, while tracheal tissue displayed an average eccentricity of 0.45, indicative of a bud-like circular tissue shape. This work highlights the robust self-organizing capabilities of human foregut cells when cultured in confined geometries and exposed to patterning signals that induce tracheal and esophageal fate, followed by region-specific morphogenesis. Capitalizing on this transformative framework that allows for large-scale reconstitution and standardization of TE development in human tissues, without wishing to be bound by theory, this work establishes a quantitative platform to uncover critical insights into human TE development and its impairment in EA/TEF.

In certain embodiments, human pluripotent stem cells (hPSCs), including embryonic stem cells (ESCs) or induced pluripotent stem cells (iPSCs), are first differentiated into definitive endoderm cells and the definitive endoderm cells are subsequently seeded onto laminin-521-coated microfabricated culture substrates to initiate foregut differentiation. The microfabricated substrates comprise elongated confinement geometries, including rectangular or elliptical adhesive domains configured to spatially restrict cell spreading and colony expansion. Definitive endoderm cells are patterned into foregut tissues by culturing in a medium containing 10 μM SB431542 and 2 μM Dorsomorphin for at least three days. The patterned foregut tissues are then cultured for approximately seven days in complete serum-free differentiation medium (SFDM) comprising Advanced DMEM/F 12, 15 mM HEPES, 2 mM L-glutamine, 1×B27 supplement, and 1×N2supplement under standard incubation conditions of 37 °C and 5% CO₂. During this period, the tissues are contacted with a lineage-directing morphogen cocktail comprising (i) 50 ng/ml KGF, 10 ng/ml BMP4, 100 nM retinoic acid, and 3 μM CHIR9902 to promote tracheal tissue determination, or (ii) 50 ng/ml KGF, 50 ng/ml Noggin, 100 nM retinoic acid, and 3 μM CHIR9902 to promote esophageal tissue determination, thereby generating tracheal or esophageal organoids (mini-organs). In some embodiments, the microfabricated culture substrate comprises a microchip configured to allow for high-throughput generation of spatially patterned epithelial tissues. In certain implementations, hPSCs are differentiated into definitive endoderm using RPMI 1640 medium supplemented with 3 μM CHIR99021 and 100 ng/mL Activin A on day 1, followed by 100 ng/mL Activin A with 0.2% deactivated fetal bovine serum (dFBS) on day 2 and 100 ng/ml Activin A with 2% dFBS on day 3 prior to seeding.

The microchip comprises micropatterned substrates that are fabricated by projection stereolithography-mediated photopolymerization of 20% 6K-8 KDa poly(ethylene glycol) diacrylate (PEGDA), 34 mM Lithium phenyl-2,4,6-trimethylbenzoylphosphinate (LAP) as photo-initiator, and 2.25 mM tartrazine as a photo-absorber to generate hydrogel microstructures directly on glass-bottom culture vessels. Digitally defined rectangular adhesive domains (100-300 μm width; ≥500 μm length) were generated by forming parallel hydrogel confinement walls surrounding coated glass regions. Following hydrogel polymerization, the substrates are coated with 10 μg/mL Laminin-521 to functionalize the exposed glass regions and support cell adhesion. Hydrogel barriers with thickness exceeding cell height are used to create well-like physical constraints that restrict stem-cell attachment to elongated geometries, enabling anisotropic tissue self-organization.

### Example 4—Morphometric Profiling Via Automated Image Segmentation and Topology Analysis

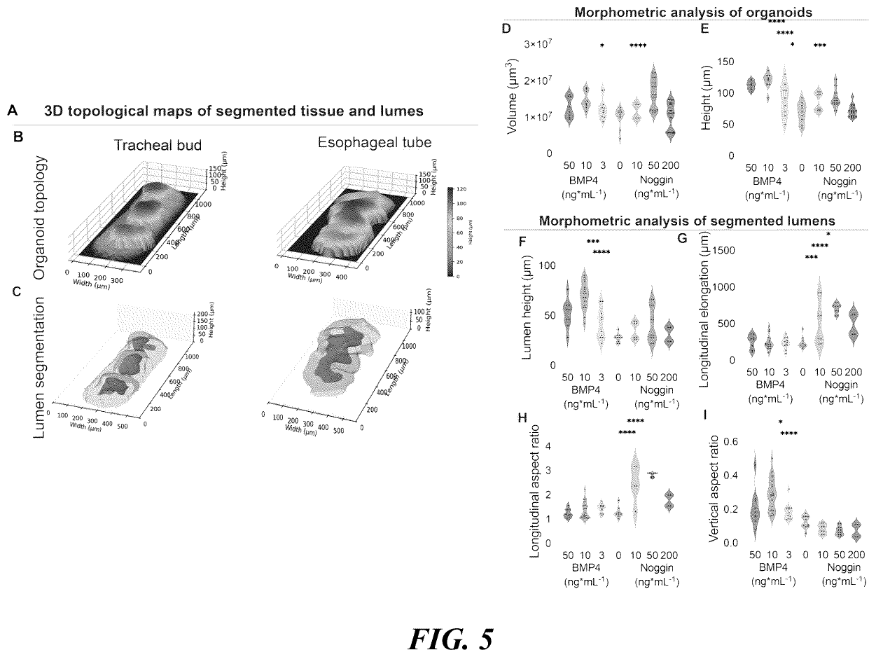

To allow for scalable and quantitative assessment of tissue-level architecture, we developed an automated machine learning-driven image analysis pipeline that segments epithelial tissues and luminal compartments and quantifies 49 morphometric parameters defining organoid and lumen structure (FIG. 5A-C), including lumen height and longitudinal elongation, as well as longitudinal and vertical aspect ratios across replicates To evaluate the capacity of our morphometric pipeline to resolve signaling-driven tissue morphogenesis, we modulated BMP activity using graded BMP4 activation or Noggin-mediated inhibition and quantitatively mapped the resulting morphogenetic responses in developing TE tissues (FIG. 5D-I). We found that intermediate BMP activity levels most effectively promoted budding morphogenesis, characterized by increased lumen height and vertical aspect ratio (FIG. 5F-I), defined as a higher height relative to the longitudinal axis of the organoid. In contrast, Noggin induced a dose-dependent shift toward tubular morphogenesis, resulting in an increased longitudinal elongation of lumens and a higher longitudinal aspect ratio, reflecting greater length relative to width with continuous, single lumens across each micropattern (FIG. 5F-I). This quantitative framework delivers precise, standardized morphometric analysis and establishes a foundation to dissect the developmental principles underlying human TE morphogenesis. To our knowledge, this is the first in vitro system that can quantitatively capture morphogenetic programs of human TE development and their modulation by graded morphogen signaling. This platform will allow for us to decode how spatial confinement and morphogen gradients synergize to direct TE morphogenesis, offering a transformative system to reveal how their dysregulation drives congenital malformations such as EA/TEF.

The tissue and organoid volume masks are generated through a hierarchical 3D morphological refinement pipeline beginning with intensity-based hysteresis thresholding as well as machine learning-based segmentation based on random forest classifiers (scikit.learn), followed by weak-zone-restricted 3D opening and small-object removal, core-constrained per-slice 2D closing and 8-connected hole filling to repair intracellular gaps, core-limited 3D micro-closing to seal discontinuities, erosion with largest-connected-component selection and re-dilation to enforce global coherence, and final volumetric smoothing; this refined tissue mask is then converted into a watertight organoid volume through planar small-hole reconstruction, basal-band patching, and 6-connected 3D face-based filling, enabling quantitative extraction of global geometric and compositional metrics including total voxel count, organoid volume, tissue volume, lumen volume, tissue and lumen fractions, surface area, convex hull area and volume, convexity, compactness, surface-to-volume ratio, apical (luminal) surface area, bounding-box dimensions (X, Y, Z), footprint area, mean and 95th-percentile thickness derived from anisotropic distance transforms, total lumens detected and retained, largest lumen fraction, depth statistics (mean, P90, weighted mean), and orientation-weighted budding metrics based on principal-axis alignment.

Lumens are defined as the volumetric difference between the watertight organoid mask and the refined tissue mask, then segmented via 3D connected-component labeling and filtered using adaptive thresholds based on relative volume (fraction of the largest lumen and fraction of total organoid volume) and anisotropy constraints that remove flattened cavities using a Z-to-XY bounding-box ratio criterion, after which retained lumens are relabeled and quantified using true 3D mesh geometry and distance-field analysis to compute voxel count, physical volume, surface area, surface-to-volume ratio, convex hull volume and area, solidity, convexity, sphericity, vertical and horizontal aspect ratios, principal-axis orientation (Z-component magnitude), centroid position in absolute and normalized coordinates, centrality relative to the organoid centroid, mean and minimum lumen depth from the outer surface, and volume-weighted summary descriptors.

In addition, the pipelines implements integrated 3D rendering and height-based visualization analysis in physical (μm) coordinates by extracting marching-cubes surface meshes for organoid and lumen structures, transforming voxel coordinates into world space with anisotropy correction, computing flat-shaded lighting (ambient-diffuse model with optional specular component), enforcing view-direction-consistent face sorting, and generating synchronized physical and height-coded renderings; a GPU-accelerated barycentric triangle rasterizer constructs Z-buffer-based top-view height maps for organoid-only and lumen-only projections, ensuring exact correspondence between 3D surfaces and 2D orthographic maps, while shared colormap normalization allows for quantitative height comparisons across views, thereby coupling morphological segmentation with geometrically faithful 3D surface analysis and synchronized 2D height-field representations for rigorous structural validation.

### Example 5-Quantitative Pipeline for Tissue Self-Organization in Human TE Morphogenesis

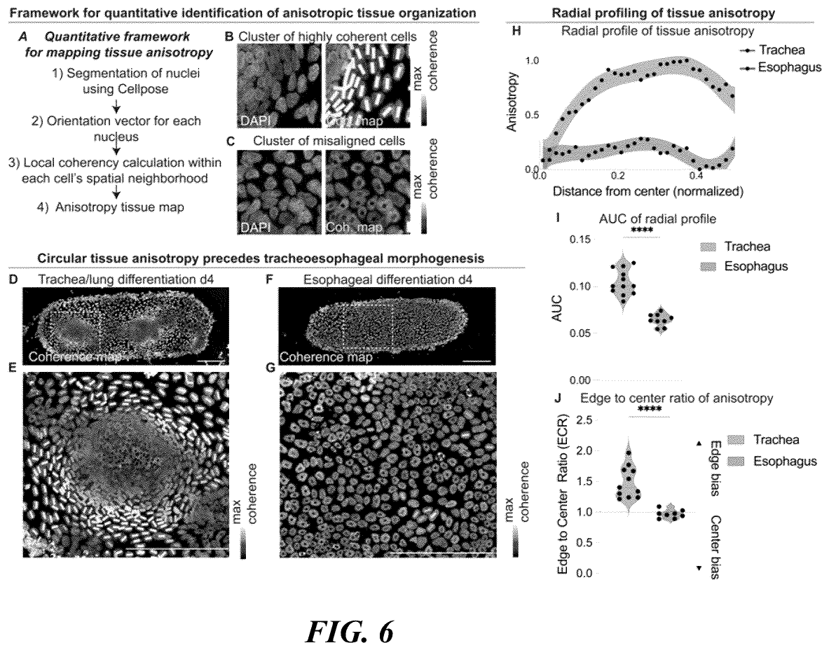

Despite its fundamental role in TE development, how tissue patterning and morphogenesis are dynamically integrated during organogenesis remains poorly understood and technically challenging to probe in vivo. We hypothesized that the trachea and esophagus exhibit distinct tissue self-organization behaviors reflecting their divergent morphogenetic outcomes. To test this, we employed vector field analysis to identify regions of tissue alignment and coherence, quantifying structural anisotropy (i.e., directional bias in tissue organization) across the entire tissue. This approach allows for the interrogation of spatial patterns of cell alignment that underlie tissue-scale anisotropic behaviors. We adapted orientation vector field analysis for fluorescence images to quantify single-cell orientation and local coherence from segmented nuclei (FIG. 6A). This approach generated tissue anisotropy maps that revealed regions of distinct alignment and directional organization across the foregut epithelium (FIG. 6B-C). We then applied this framework to foregut cells differentiating into tracheal or esophageal lineages, collected at day 4 of differentiation. Interestingly, tracheal cells exhibited a pronounced circular pattern of tissue anisotropy encircling the nascent bud primordium, a signature absent during esophageal differentiation (FIG. 6D-G). Quantitative analysis demonstrated a radial anisotropy profile with an edge-enriched orientation bias, as reflected by an increased Edge-to-Center Ratio (ECR) (FIG. 6H-J). This analysis unveils a previously unrecognized radial self-organization of tracheal epithelia that anticipates morphogenesis, highlighting fate-driven tissue patterning as an early determinant of organ shape and compartmentalization.

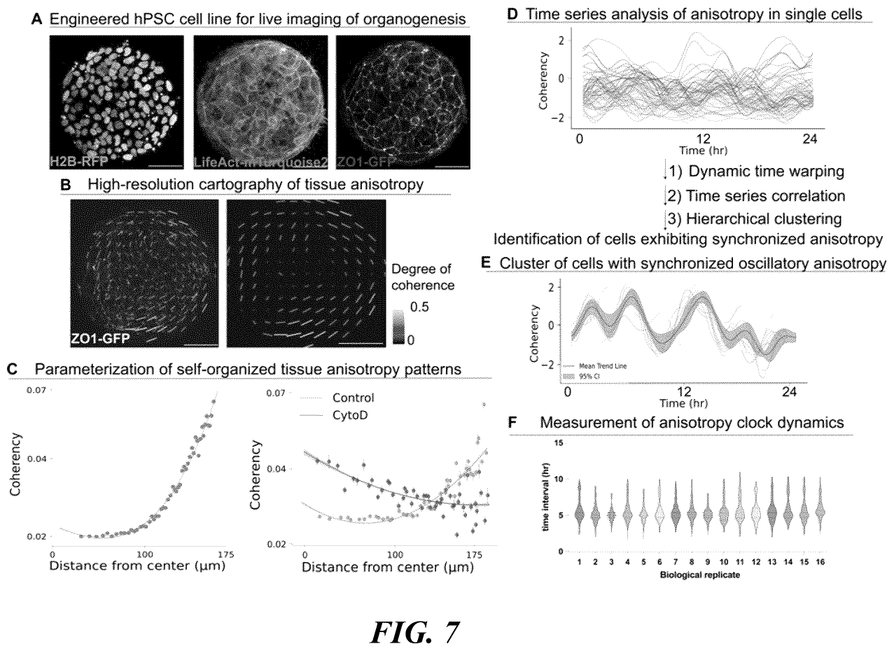

To investigate the temporal dynamics of tissue anisotropy during TE development, we integrated quantitative anisotropy analysis (FIG. 7) with live-cell confocal imaging using a hPSC line we engineered that allows for real-time visualization of tight junctions (ZO-1), F-actin (LifeAct), and nuclei (H2B), thereby capturing cellular organization throughout TE organogenesis (FIG. 7A). Consistent with analyses in fixed samples, we observed the emergence of a circular anisotropy pattern preceding bud morphogenesis (FIG. 7B-C). Inhibition of actin polymerization with Cytochalasin D abolished coordinated anisotropy, indicating that this process is driven by cell contractility-dependent behavior (FIG. 7C). We then tracked how individual cells aligned with their neighbors over time, uncovering oscillatory fluctuations in cellular anisotropy during the initial 24 hours of tracheal morphogenesis (FIG. 7D). To further investigate the dynamic regulation of tissue anisotropy, we developed a framework for quantifying anisotropy at single-cell resolution from live imaging data, which we termed Spatiotemporal Analysis of Tissue Anisotropy (SATA). We applied dynamic time warping (DTW), proximity-based correlation analysis, and hierarchical clustering to identify clusters of neighboring cells exhibiting synchronized patterns of anisotropy. This approach uncovered a previously unknown oscillatory behavior that directs tissue anisotropy during tracheal morphogenesis (FIG. 7E), which is reproducible and parameterizable across biological replicates (FIG. 7F). This time-dependent mechanism directs periodic alignment of neighboring cells, with peripheral orientation peaking at ~5-hour intervals (FIG. 7D-F). We will capitalize on this analytical toolkit to reveal the temporal logic of supracellular self-organization, mapping the dynamic evolution of tissue anisotropy during TE development and how developmental cues converge to direct this process.

### Example 6—Single Cell RNA-Sequencing of Trachea-Esophageal Mini-Organs

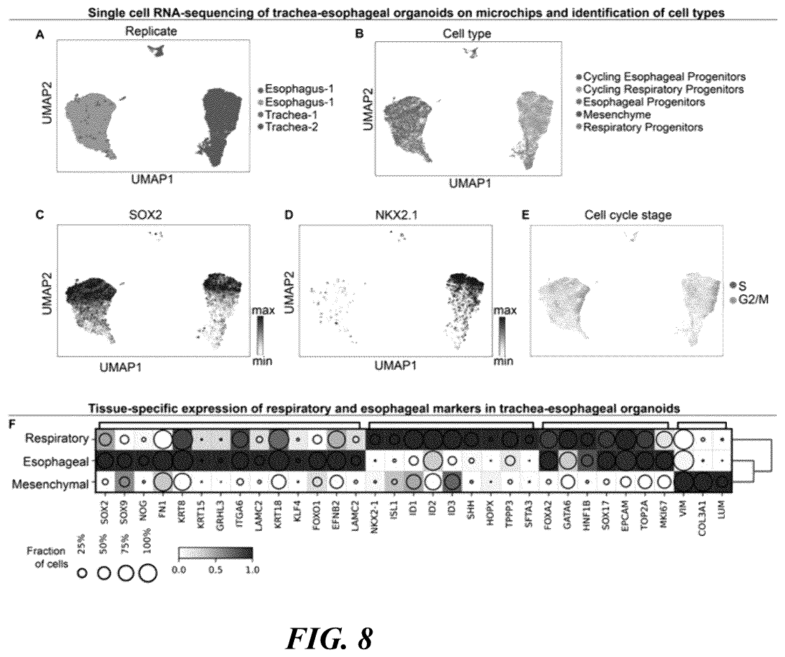

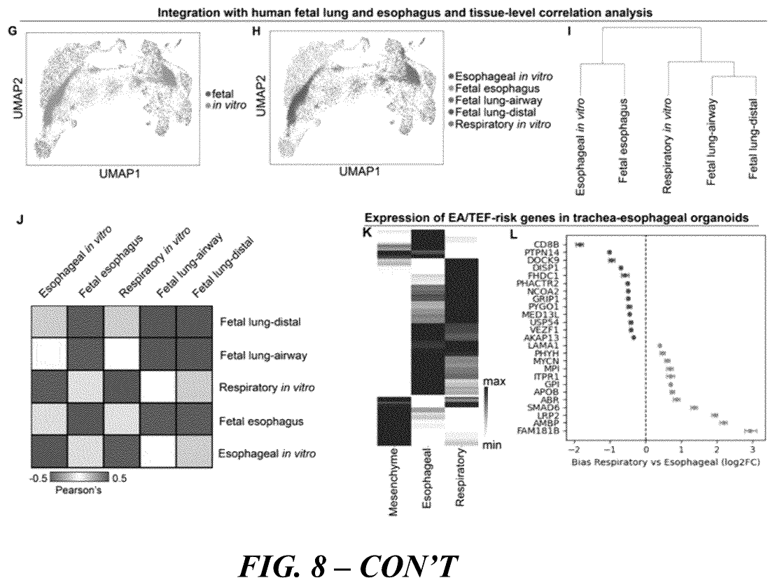

To characterize cellular complexity and gene expression programs underlying human tracheoesophageal (TE) development, we performed single-cell RNA sequencing of TE organoids generated across two biological replicates of tracheal and esophageal tissues at day 7 of morphogenesis, which showed high concordance in cellular composition (FIG. 8A). Unsupervised analysis identified five major cell populations, including cycling and non-cycling esophageal and respiratory progenitors as well as mesenchymal cells (FIG. 8B). Cycling progenitors displayed enrichment of genes associated with S-phase and G2/M cell-cycle programs (FIG. 8E). Consistent with developmental identity, SOX2 expression was enriched in esophageal cells and present at lower levels in respiratory progenitors (FIG. 8C), whereas NKX2.1 expression was restricted to respiratory organoids (FIG. 8D). Lineage-specific profiling revealed enrichment of esophageal markers (SOX2, SOX9, NOG, KRT8, KRT15, ITGA2, and FOXO1), respiratory markers (NKX2.1, ISLI, IDI, SHH, HOPX, TPPP3, and SFTA3), and mesenchymal markers (VIM, LUM, and COL3A1), while genes associated with epithelial foregut identity were shared across both epithelial compartments (FIG. 8F).

To assess developmental fidelity, we integrated in vitro TE organoids with published single-cell atlases of human fetal lung and esophagus at post-conception weeks 11-15¹, representing the earliest available reference datasets for TE organs. in vitro-derived esophageal and respiratory cells mapped preferentially to their respective fetal counterparts (FIG. 8G-H), displaying strong tissue-specific transcriptional correlation, whereas cross-tissue comparisons showed anticorrelation, supporting lineage specificity (FIG. 8I-J). To evaluate disease relevance, we profiled expression of 247 genes associated with esophageal atresia and tracheoesophageal fistula (EA/TEF), detecting 138 genes within our dataset (FIG. 8K), including 25 exhibiting tissue-biased expression patterns (FIG. 8L). Together, these results demonstrate that TE organoids recapitulate key cellular diversity and conserved gene expression programs of the developing human TE tract while capturing disease-associated genetic signatures. This reference atlas establishes a foundational framework by (1) validating the physiological relevance of our model through comparison with human fetal tissues, (2) demonstrating the feasibility of single-cell approaches to resolve tissue-specific developmental programs, and (3) enabling mechanistic investigation of EA/TEF pathogenesis.

### Example 7—Modeling EA/TEF on Micropatterned TE

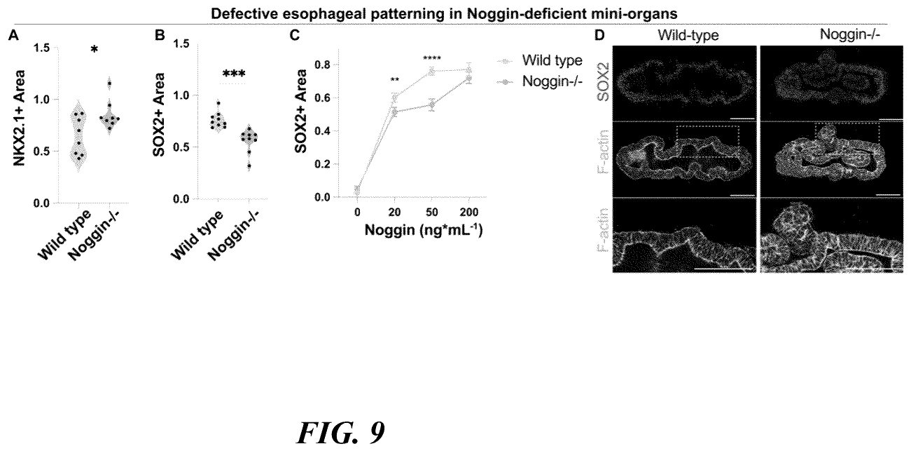

Without wishing to be bound by theory, deregulated activity of key developmental signaling pathways contributes to EA/TEF. One of such pathways is the BMP pathway, which plays a significant role in TE development and its disruption in model organisms mimics key pathophysiological features observed in patients with EA/TEF. BMPs are secreted proteins that bind to BMP transmembrane receptors, which in turn regulate downstream SMAD-mediated transcriptional activity. This signaling is regulated by extracellular antagonists, such as Noggin, which bind BMP ligands to prevent receptor activation and inhibit BMP pathway activity. Notably, the majority of EA/TEF patients exhibit a loss of Noggin expression in the esophagus, where it is present. BMP hyperactivation, as a consequence of loss of Noggin, contributes to esophageal immaturity and abnormal development of respiratory glands within the esophagus, through currently unknown mechanisms. To further validate the role of this gene in TE development, we utilized a Noggin-knockout hPSC line (Noggin−/−) to generate micropatterned TE mini-organs. Interestingly, Noggin−/− cells exhibit incomplete esophageal patterning, even in response to high concentrations of exogenous Noggin (FIG. 9A-D), as observed in EA/TEF patients. Noggin loss also led to an increase in NKX2.1+ tracheal cells in absence of patterning signals (FIG. 9A-D). Concomitantly, Noggin−/− organoids exhibited defects in esophageal morphogenesis, characterized by the formation of aberrant bud-like glandular structures (FIG. 9D), features observed in EA/TEF patients. These findings indicate that a coordinated balance of BMP activation and inhibition is essential for proper TE patterning, and that disrupting this balance skews cell fate toward the tracheal lineage. Describe herein are experiments that elucidate how the BMP pathway orchestrates TE fate and morphogenesis, and uncover how its deregulation leads to biased fate acquisition, contributing to the pathogenesis of EA/TEF.

### Example 8

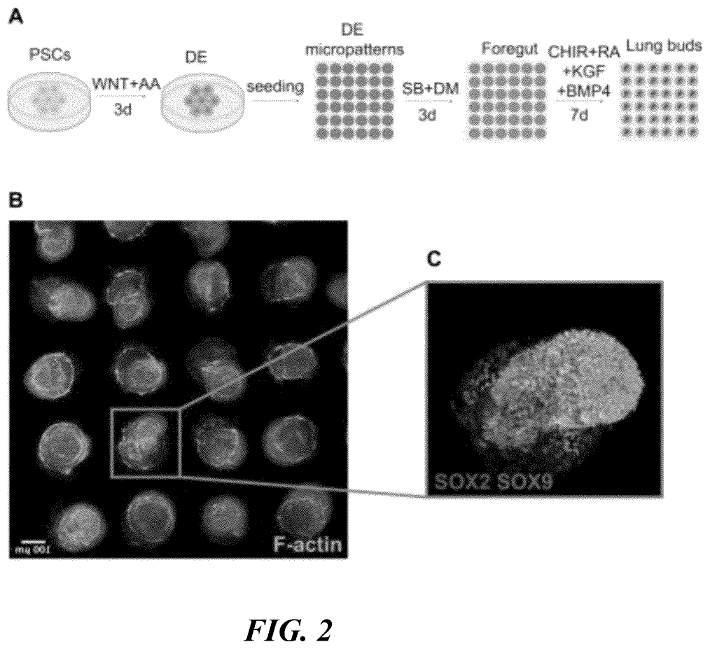

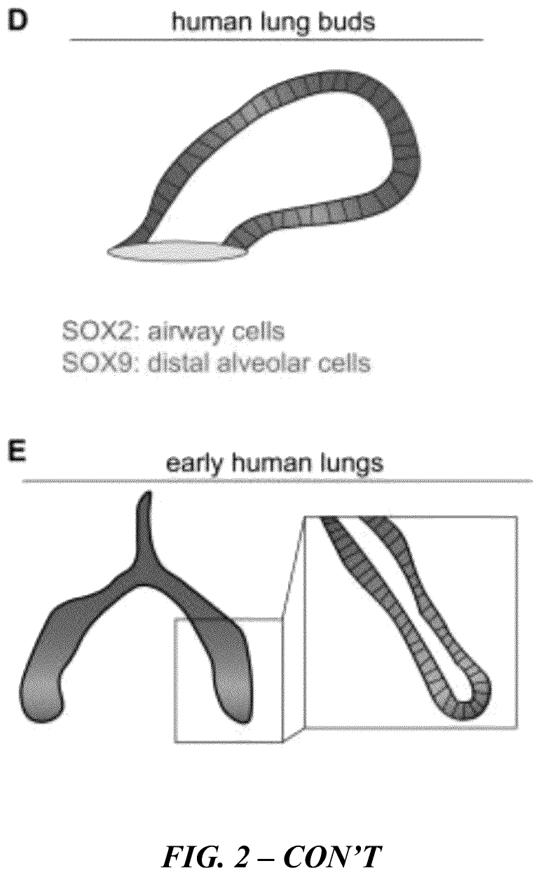

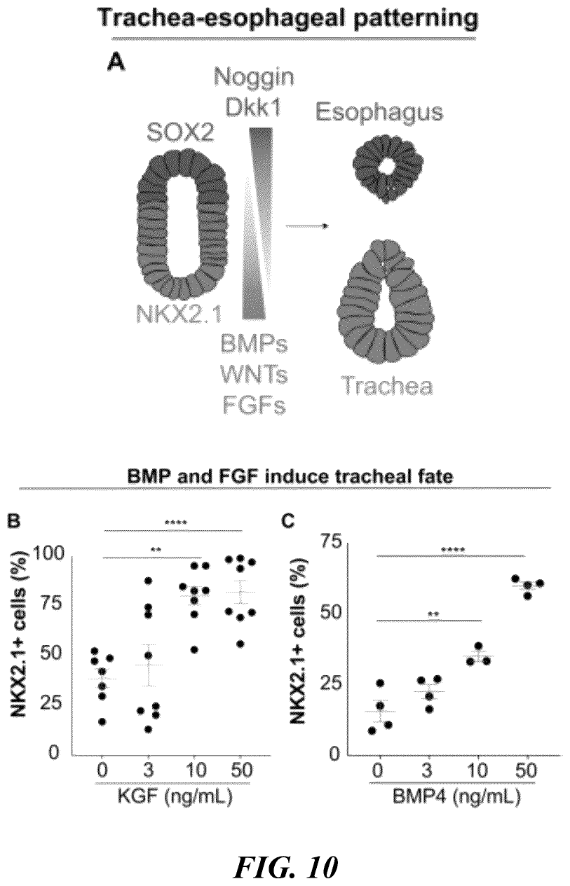

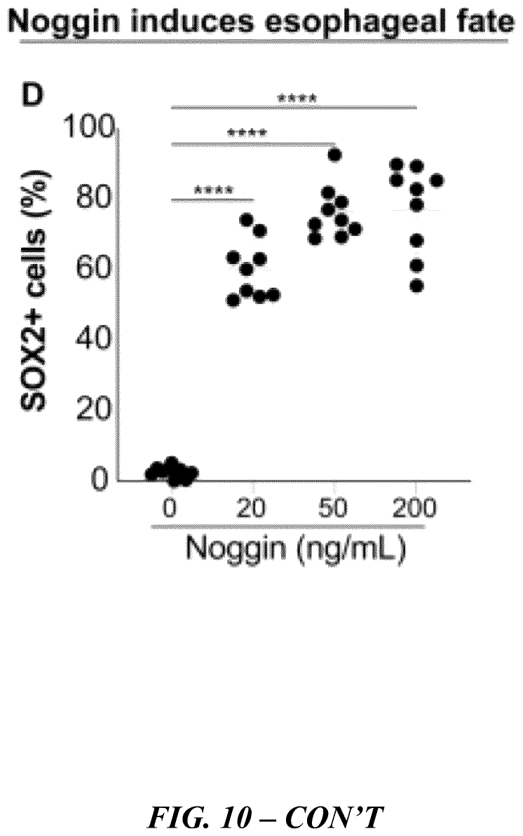

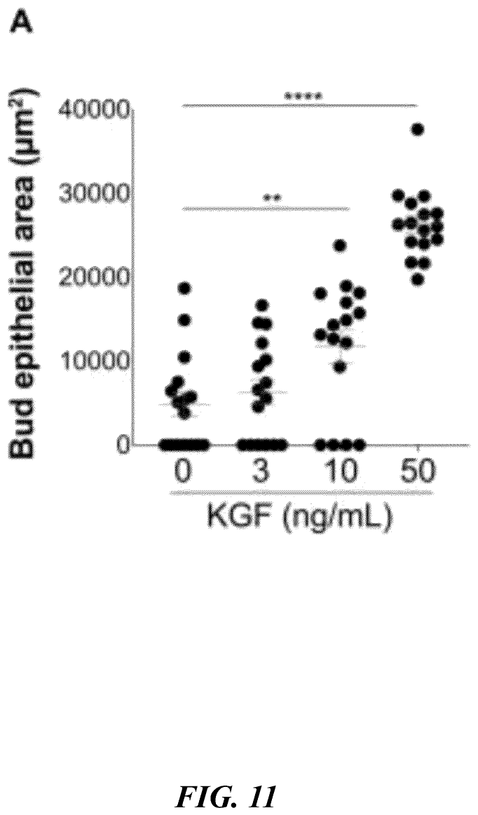

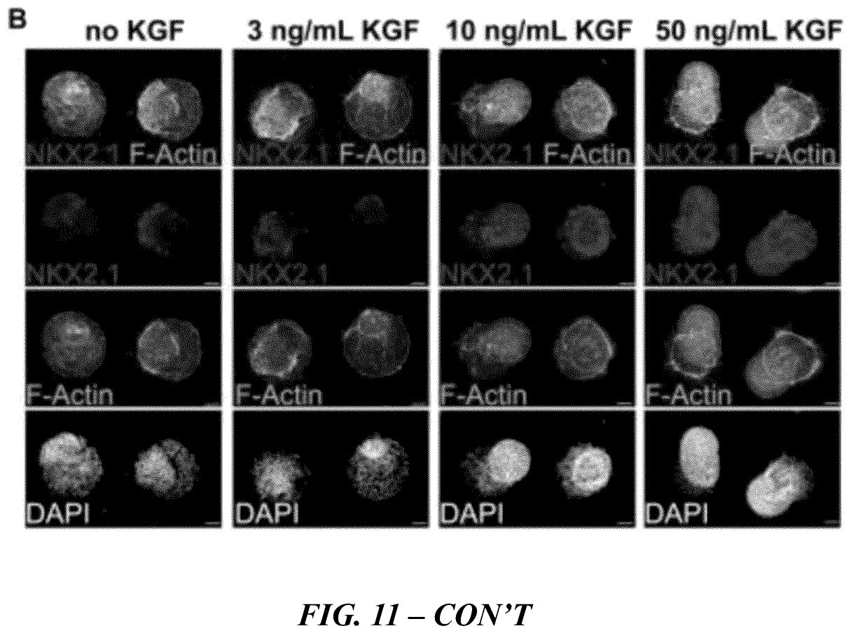

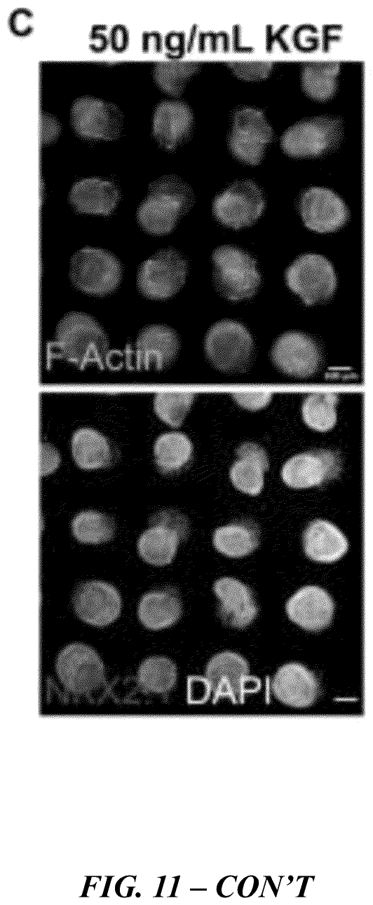

During TE development, the precursor foregut tube becomes regionally patterned along the D-V axis by a core group of signaling pathways including BMPs, WNTs, and FGFs (FIG. 10A). These pathways engage in reciprocal interactions that coordinate D-V patterning and morphogenesis. These processes collectively determine the fate and architecture of the trachea and esophagus, yet the mechanisms underlying their regulation remain largely unknown. Interestingly, we have found that human tracheal bud morphogenesis in vitro relies on the combined modulation of WNT, BMP4, and KGF signaling (FIG. 2A). Among these, KGF and BMP4 signaling levels are critical, with dose-dependent increases in their activity significantly enhancing the differentiation of NKX2.1+ tracheal progenitors (FIG. 10B-C). High levels of KGF signaling specifically drive tracheal bud morphogenesis, producing tracheal buds of commensurate size and morphology with high efficiency (FIG. 11). Conversely, Noggin induces patterning of foregut cells into SOX2+ esophageal progenitors at the expense of tracheal cells (FIG. 10D). Together, these results demonstrate that modulation of BMP and FGF pathways steers tracheal versus esophageal fate and region-specific morphogenesis in vitro (FIG. 11).

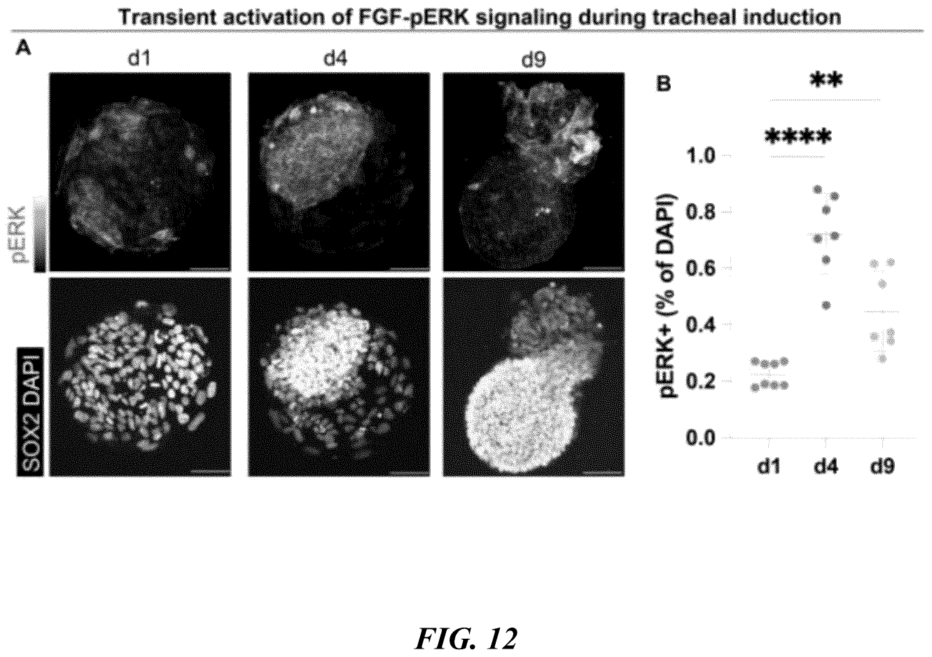

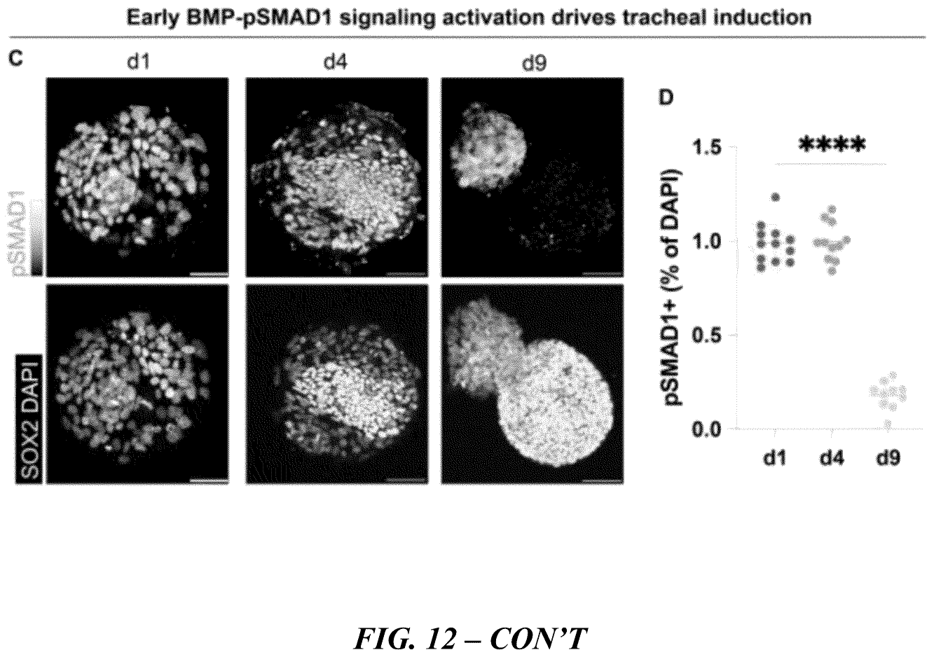

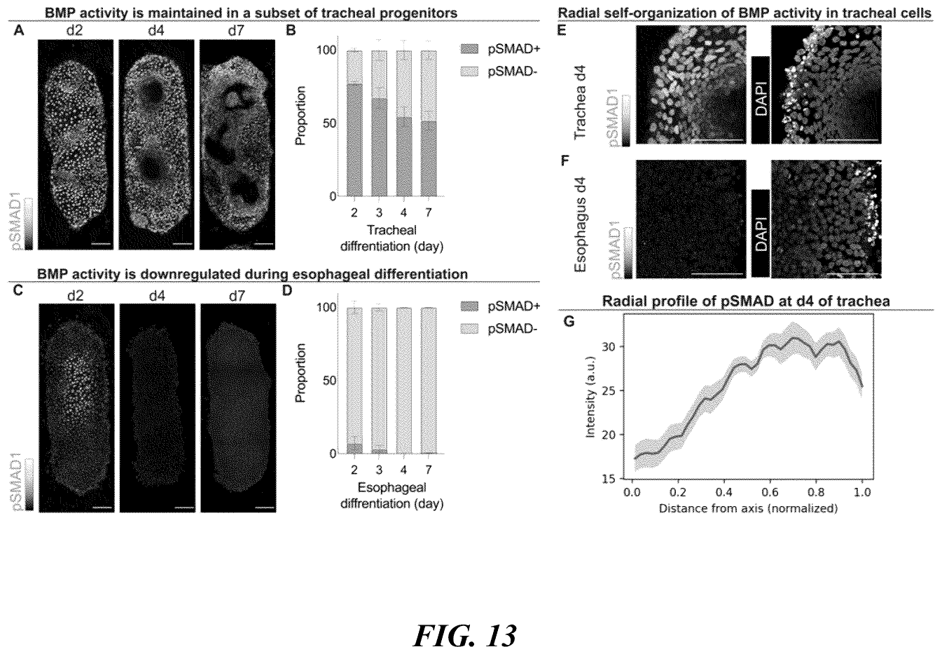

Both these pathways exhibit time-dependent signaling activities in differentiating foregut cells in vitro. Based on phospho-ERK (pERK, T202/Y204) immunostaining as a proxy for FGF signaling activity, FGF activity is highly induced in SOX2+ esophageal cells in early stages of TE patterning at day 4 and then becomes downregulated (FIG. 12A-B). We observed that phospho-SMAD1(pSMAD1, S463/465), a proxy for BMP pathway activation, is differentially regulated during TE development (FIG. 13A-D). High levels of BMP activity were observed in early stages of tracheal bud fate patterning at days 1 and 4 (FIG. 12C-D). While esophageal cells displayed downregulation of BMP activity, tracheal cells maintained robust BMP activity throughout the time-points analyzed (FIG. 13A-D). Interestingly, BMP signaling displays a circular pattern during tracheal differentiation, with activity concentrated at the colony periphery (FIG. 13E-G), mirroring the tissue anisotropy profile observed in this lineage (FIGS. 6 & 7). We hypothesize that BMP signaling modulates anisotropy dynamics to coordinate the morphogenetic program of TE development. Thus, without wishing to be bound by theory, time-dependent regulation of these pathways is critical for TE patterning and morphogenesis. This study underscores the power of the human TE micropattern model as a paradigm to uncover the roles of developmental signaling in this process. Accordingly, how FGF and BMP signaling pathways orchestrate fate specification and morphogenesis in the human TE, as well as in EA/TEF, will be validated.

### Example 9

EA/TEF arises from disrupted developmental signaling and associated cell identity crises during TE development, yet the precise mechanisms underlying these defects remain poorly understood. Dysregulation of the BMP pathway, a key regulator of fate patterning and morphogenesis, appears to play a central role. Notably, loss of the BMP inhibitor Noggin has been implicated in EA/TEF, with a reduced expression of Noggin in the esophageal tissues of patients. As described herein loss of Noggin promotes EA/TEF-like phenotypes (FIG. 9), and, without wishing to be bound by theory, this loss drives skewed fate bias towards tracheal lineages, and defective patterning into esophageal tissue due to deregulated signaling and gene expression dynamics.

### Example 10—To Unveil TE Defects by EA/TEF-Associated Genes Through Unsupervised Phenotypic Profiling

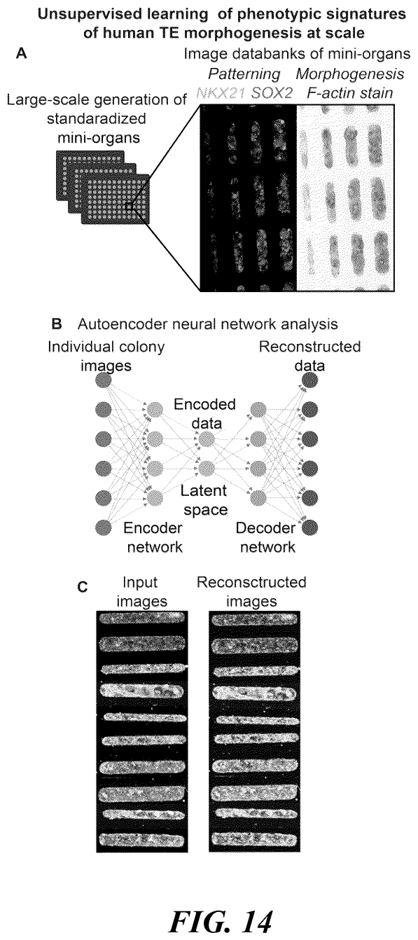

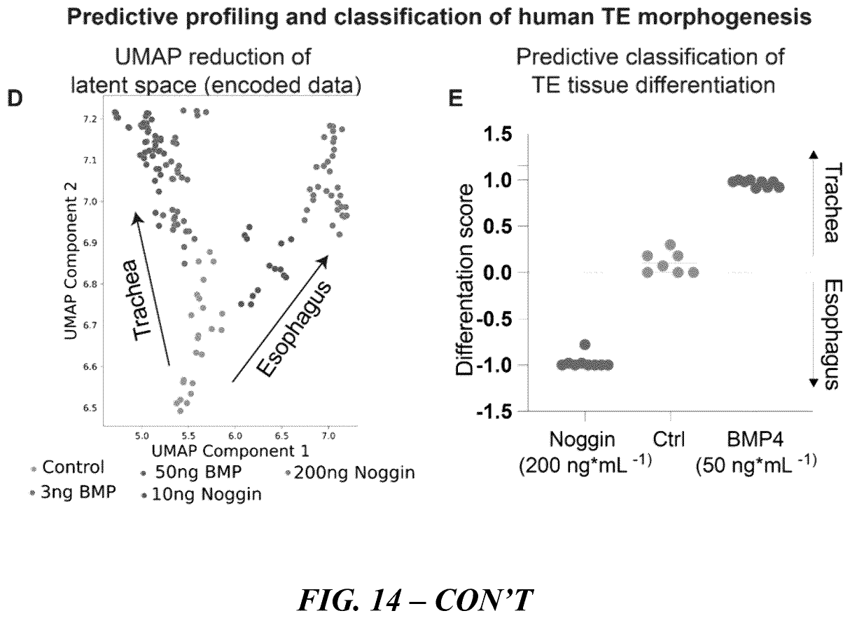

A key challenge in utilizing organoid technologies to study the developmental mechanisms governing TE formation lies in the lack of standardization due to the intrinsic variability of multicellular processes and the high degree of structural heterogeneity among organoids. To allow for phenotypic analysis of human organ development and disease at scale, a high-throughput platform for generating micropatterned mini-organs has been developed (FIG. 14A). This standardized system produces organotypic tissues in a multi-well plate format, with each well containing hundreds of self-organizing and commensurate hPSC colonies (FIG. 14A). This platform can be leveraged for high-throughput tracking of SARS-COV-2 infection, identifying therapeutic targets through candidate-based and arrayed CRISPR genetic screening. To allow for artificial intelligence (AI)-based unsupervised learning and predictive classification of TE development, image databanks of micropattern colonies labeled with TE patterning and morphogenesis markers were developed as input for autoencoder neural network analysis (FIG. 14A-C). Autoencoders are designed to compress input data into a lower-dimensional latent space, an abstract representation that captures the most critical features of the data, which can be used to reconstruct organoid images with high efficiency using decoder networks (FIG. 14C). We developed a deep convolutional autoencoder to learn compact, biologically meaningful representations of RGB microscopy images while preserving fine structural and color information required for quantitative phenotypic analysis. The encoder is based on a pretrained ResNet50 architecture, which progressively extracts hierarchical visual features ranging from low-level texture and edge information to high-level tissue architecture through sequential residual blocks. The deepest encoder features are compressed using a 1×1 convolutional bottleneck into a reduced latent feature space that retains spatial organization, enabling efficient encoding of organoid morphology without collapsing structural relationships into a single vector representation.

Image reconstruction is achieved through a symmetric decoder incorporating U-Net-style skip connections that directly transfer multi-scale spatial information from encoder layers to the reconstruction pathway, allowing accurate recovery of epithelial boundaries, lumen geometry, and subcellular texture. Instead of transposed convolutions, the decoder employs bilinear upsampling followed by convolutional refinement to minimize checkerboard artifacts observed in generative reconstruction networks. The final reconstruction layer uses a Tanh activation rescaled to the [0,1] intensity range and includes a learnable per-channel gain parameter, enabling adaptive correction of channel-specific intensity imbalance and improving fidelity across fluorescent or pseudocolor imaging channels.

Training optimizes a composite reconstruction objective combining perceptual loss derived from intermediate VGG16 feature representations, pixel-wise L1 reconstruction loss, and channel-weighted color loss to jointly preserve global morphology, local structural detail, and color composition. This multi-objective strategy promotes reconstruction of biologically relevant tissue architecture rather than simple pixel similarity. Reconstruction quality is continuously evaluated using structural similarity index (SSIM) and peak signal-to-noise ratio (PSNR), while latent representations are globally summarized via adaptive pooling and visualized using UMAP dimensionality reduction to monitor emergence of phenotypic structure within the learned embedding space during training. Together, this framework allows for artifact-suppressed image reconstruction while simultaneously generating an interpretable latent space suitable for downstream morphological classification and phenotypic discovery.

The latent features were analyzed using UMAP dimensionality reduction, generating a graph-based phenotypic landscape that captures the diversity of TE development (FIG. 14D). This approach revealed subtle variations in TE development, such as dose-dependent effects of BMP4 and Noggin, which allowed for the efficient segregation of differentiation phenotypes across experimental conditions (FIG. 14D). A random forest classifier was then deployed to predict TE differentiation phenotypes, building on methods for unbiased predictive classification of high dimensional biological datasets using AI. This classifier accurately predicted the impact of BMP4 and Noggin on their respective effects on tracheal and esophageal differentiation, respectively (FIG. 14E). The predictive algorithm demonstrated remarkable robustness, with a coefficient of variation below 10% across biological replicates and a Z′ score of 0.96, exceeding the benchmarks for high-throughput assays. This framework facilitates unbiased phenotypic analysis for classifying and predicting developmental trajectories at scale, which will provide new insights into the mechanisms underlying TE development and EA/TEF.

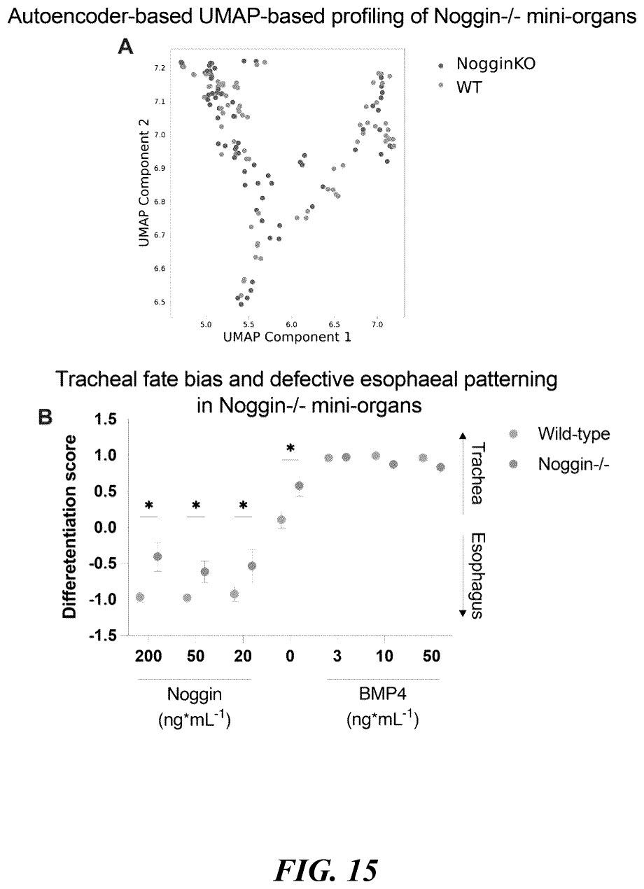

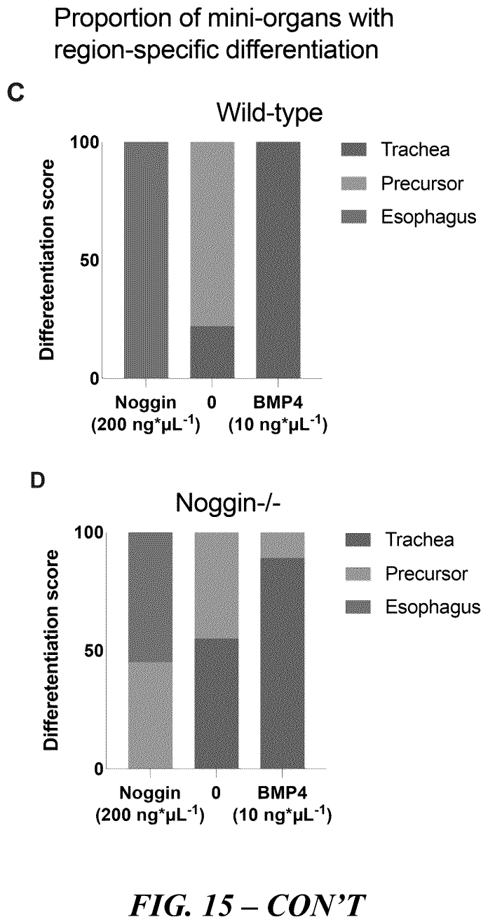

The autoencoder-based unsupervised phenotypic profiling approach was employed to analyze image databanks of Noggin−/− TE mini-organs (FIG. 9), which display key hallmarks of EA/TEF. We mapped these data onto wild-type samples (FIG. 15A) and then measured differentiation probabilities using the random forest classifier analysis. Consistent with EA/TEF features in patients, analysis revealed that Noggin−/− cells predominantly exhibited an immature phenotype, with 1) a low esophageal differentiation score, 2) precursor-like characteristics, and 3) a bias toward a tracheal-like phenotype in the absence of patterning signals (FIG. 15B-D). Mimicking findings in EA/TEF patients, these analyses indicate that EA/TEF-associated genetic insults can shift cellular competence, resulting in biased fate outcomes during TE differentiation. Without wishing to be bound by theory, we can systematically test this by applying these predictive models to other EA/TEF-associated genetic perturbations, to determine whether this represents a generalizable mechanism underlying organ malformations that originate as a consequence of disrupted TE development.

### Unsupervised Phenotypic Profiling of Tissue Morphogenesis

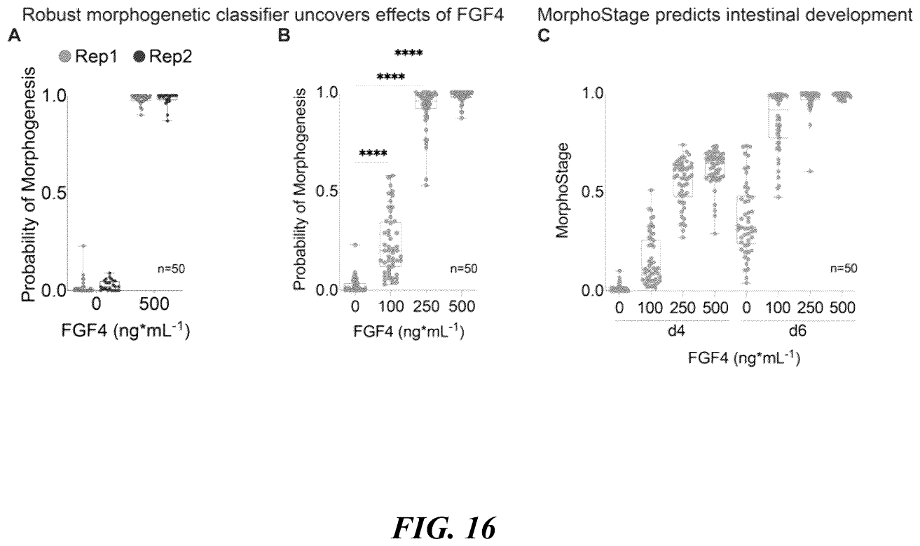

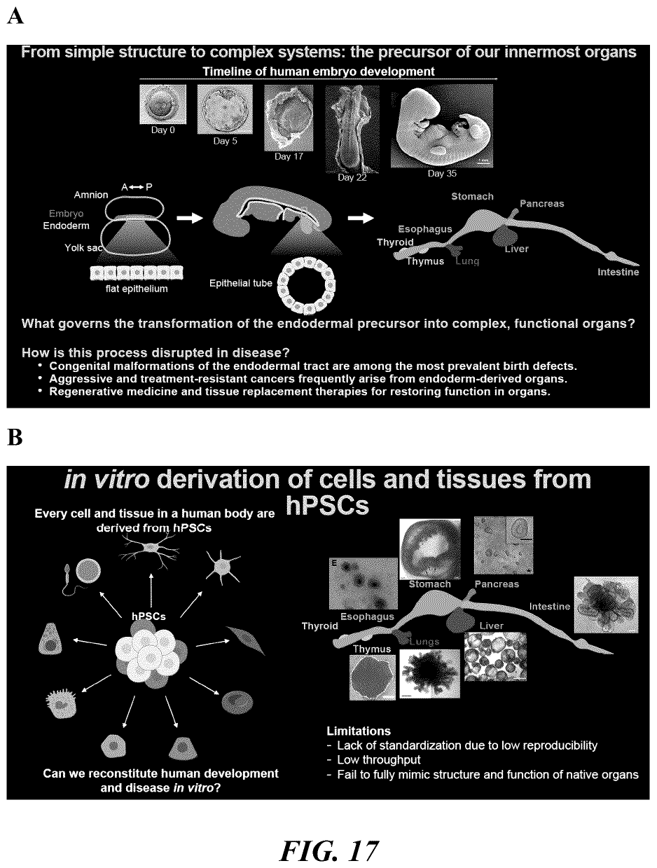

We implemented a random forest classifier to predict tissue morphogenesis phenotypes (FIG. 16). This model robustly and accurately predicted the effects of FGF4 on villus morphogenesis across doses and biological replicates (FIG. 16A-C), highlighting its utility for phenotypic classification and dose-responsiveness profiling of tissue morphogenesis. We then trained a random forest regressor to build MorphoStage, a quantitative metric designed to track phenotypic progression during tissue morphogenesis, sensitive to both temporal dynamics and morphogen modulation. This algorithm accurately predicted the stage of morphogenesis across varying FGF4 doses and timepoints (FIG. 16C), revealing a continuous, dose-dependent trajectory of villus development. The algorithm demonstrated remarkable robustness, with a coefficient of variation below 10% and a Z′ score >0.5. This framework allows for unbiased, predictive phenotypic profiling of human organoids, offering a powerful platform to accelerate therapeutic testing and discovery.

### Example 11

We will perform an arrayed CRISPR screen of 35 genes identified as having EA/TEF causative mutations in patients (CHD7, EFTUD2, ERCC4, FANCA, FANCB, FANCC, FAND1, FANG, FBN2, FGFR2, FLNA, FOXF1, FREM2, GDF3, GDF6, GLI3, MDM2, MEOX2, MID1, MKKS, MYCN, NKX2-1, NOG, PLEC1, PTEN, RARA, RARB, RBM8A, RIPK4, RMRP, SEMA3E, SMARCD1, SOX2, SOX4, SPECC1L, TBX1, TCOF1, WBP11, YY1, ZIC3). Many of these genes are linked to syndromes with TE defects, such as CHARGE syndrome (CHD7), Fanconi anemia (FANCA-G), Feingold syndrome (MYCN), and VACTERL association (SOX2, ZIC3)1-5. Another subset of genes (FOXF1, TBX1, FGFR2, BMP4, BMPR1A/B, GLI3, NOG, PTEN, and others) are regulators of the BMP, WNT, SHH and FGF pathways, which are critical for TE development.

### Example 12—Reconstitution of Human Trachea-Esophageal Development and Disease at Scale

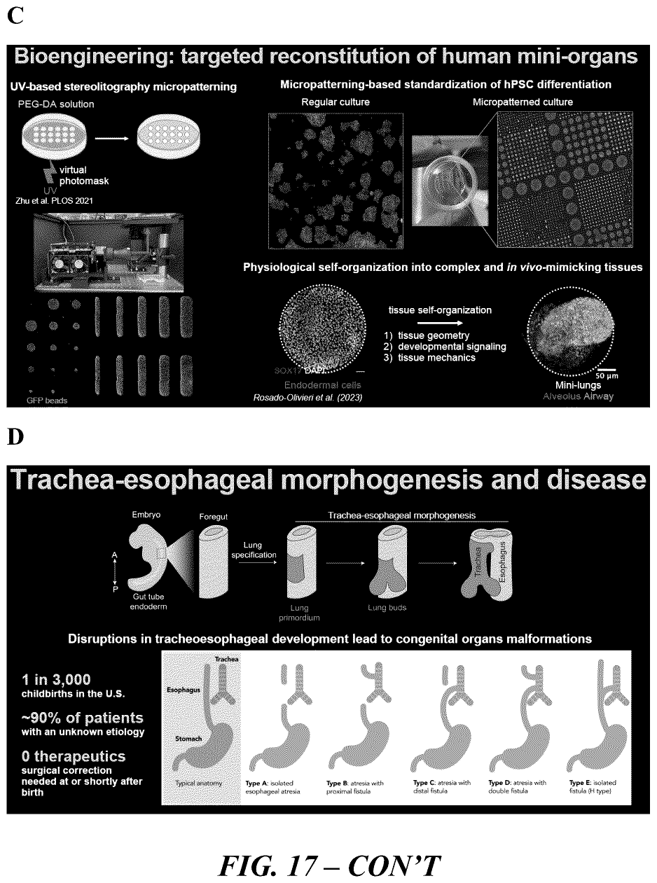

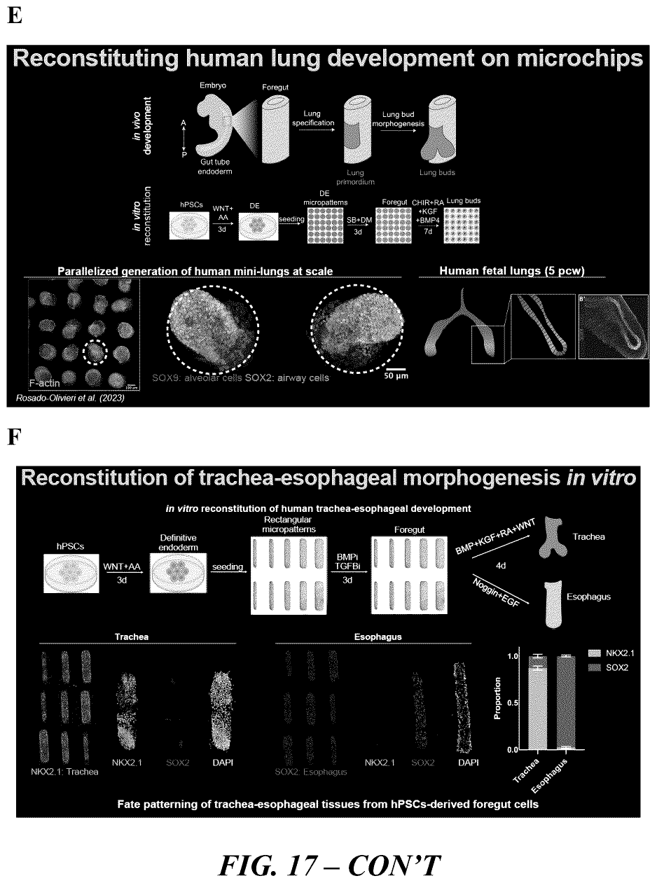

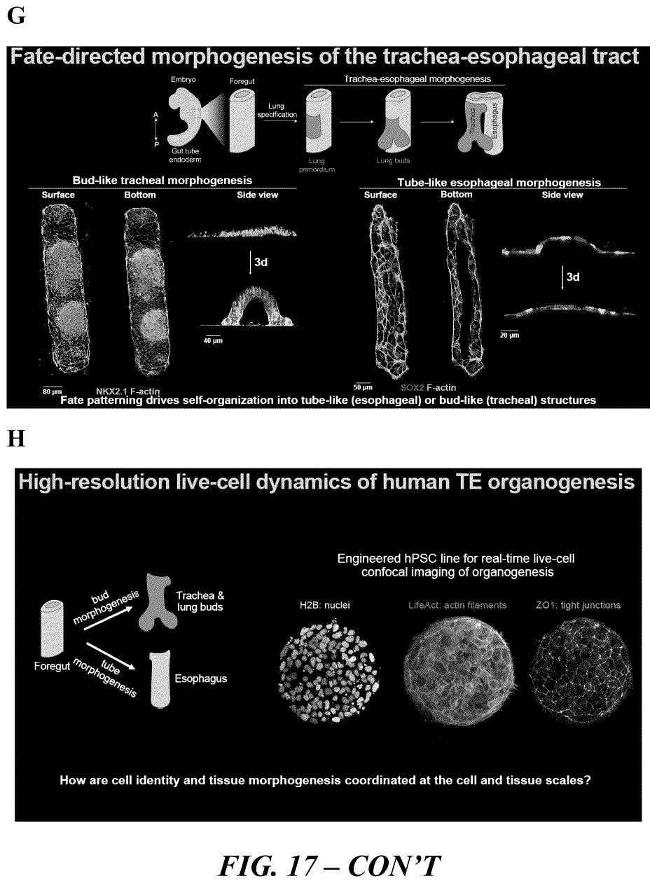

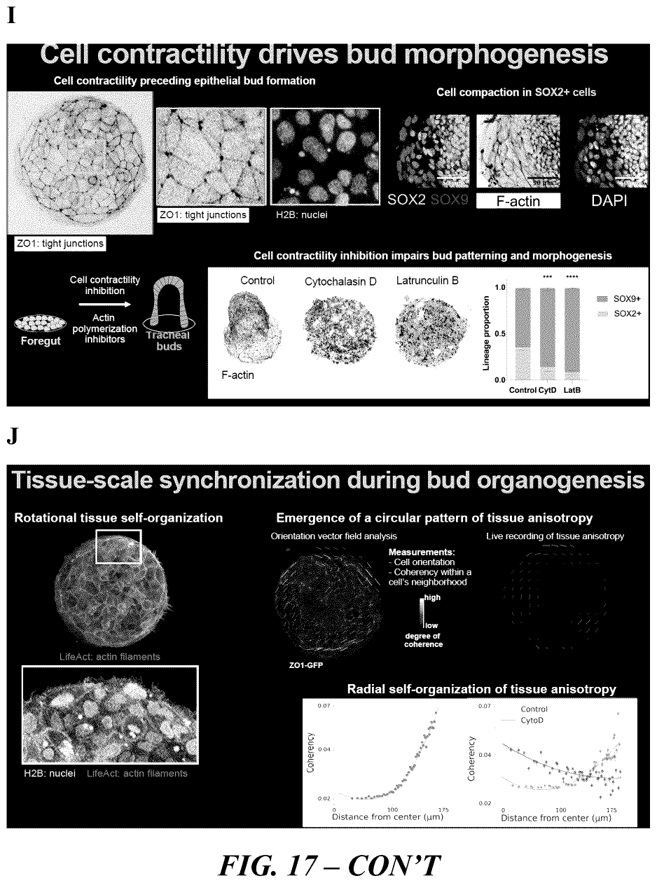

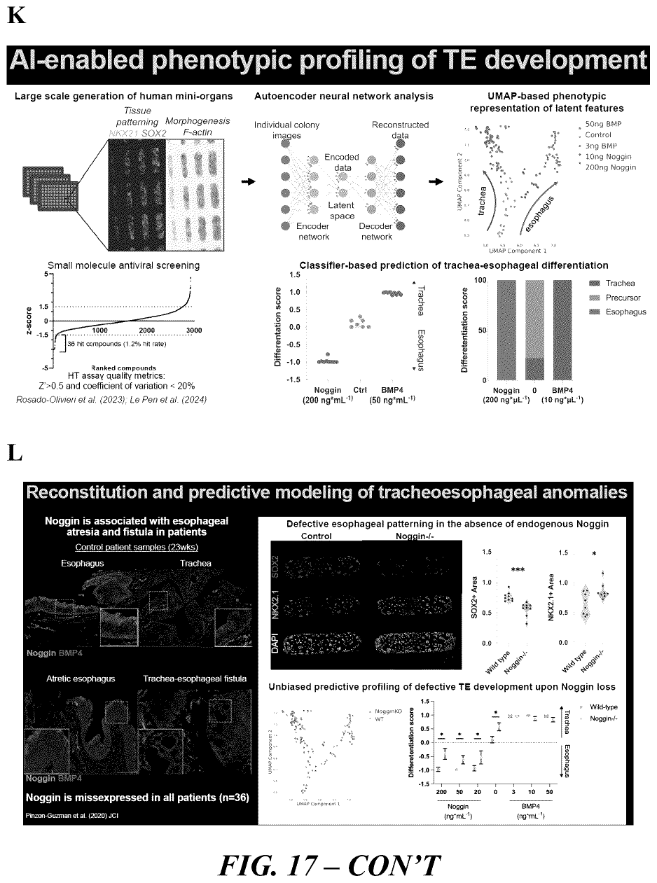

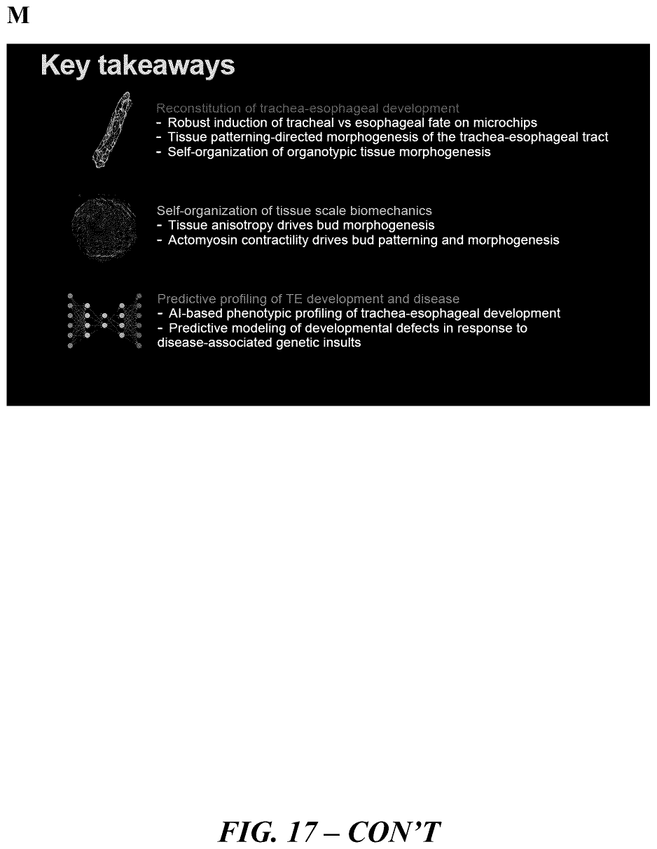

See also FIG. 17.

We are interested in elucidating the remarkable journey of how the human body's innermost organs originate from a fertilized egg during human embryo development. Starting from Day 0 with the fertilized egg, we move through key stages of early human embryonic development—by Day 5, the blastocyst forms; by Day 17, gastrulation begins, establishing the germ layers. By Day 22, the primitive endodermal tube emerges, and by Day 35, we start to see the complex morphology of a developing embryo. During this process, we see the transformation from a flat epithelium of endodermal cells into a three-dimensional epithelial tube. This tube is the developmental precursor to endoderm-derived organs. (FIG. 17A) From this simple tube arise the major components of the endodermal tract—esophagus, stomach, intestines—as well as vital organs like the liver, pancreas, lungs, thyroid, and thymus. Each region becomes specified, patterned, and morphogenetically sculpted into a distinct organ system. (FIG. 17A)

A central question in developmental biology is: What governs the transformation of this uniform endodermal precursor into such a diverse array of functional organs? (FIG. 17A)

Understanding this process is not only fascinating from a basic science perspective—it's critical for medicine. Congenital defects of the endodermal tract are among the most common birth defects. Many aggressive and treatment-resistant cancers arise from these tissues. And insights into this transformation are key to developing regenerative therapies for endodermal organs. (FIG. 17A)

To better understand this process, our work harnesses human pluripotent stem cells, as they have the remarkable potential to differentiate into every cell and tissue type in the human body. (FIG. 17B)

From a single pluripotent population, we can derive neurons, blood cells, muscle, epithelial cells, and more. This capacity makes hPSCs an indispensable tool for modeling human development, disease, and regeneration. (FIG. 17B)

Over the past decade, we have worked to harness this potential to generate organoids-miniaturized, three-dimensional models of human organs. Here, you can see organoids that mimic different segments of the endodermal tract, including the stomach, intestine, liver, pancreas, lungs, and more. These organoids often recapitulate key features of human tissue architecture and function, offering unprecedented insight into human biology. (FIG. 17B)

But the field still faces significant hurdles. A key challenge remains the lack of standardization—many protocols suffer from low reproducibility and throughput. This limits broader adoption and translational application. (FIG. 17B)

Our goal is to address these limitations to guide more faithful and efficient in vitro organogenesis. (FIG. 17B)

The central question driving much of our ongoing research: How can we faithfully recapitulate human organ development in vitro? (FIG. 17B)

To overcome the challenges of current organoid methods, we've integrated a bioengineering approach to enhance the precision and reproducibility of organoid generation. (FIG. 17C)

Using UV-based stereolithography, we can project custom virtual photomasks onto a hydrogel solution containing PEG-DA. This creates precise micropatterns on the substrate, guiding cell attachment and organization. (FIG. 17C)

Described in this example is the system in action—a custom-built stereolithography setup that uses projected UV light to define user-specified patterns. You can see examples of the resulting patterned features visualized with GFP beads—ranging from circular to rectangular geometries. (FIG. 17C)

When we compare regular culture to microchips, the difference is striking. In conventional settings, colonies grow irregularly and unpredictably. But with micropatterning, we achieve uniform colony size and spatial arrangement—setting the stage for standardized differentiation. This approach allows us to scale and reproduce thousands of mini-organs within a single microchip, paving the way for high-throughput quantitative analysis of human development and disease. (FIG. 17C)

Here, we show that micropatterned colonies self-organize into spatially ordered endodermal tissues, marked by SOX17 expression. And we can take this further—engineering mini-lungs that exhibit organized domains of alveolar and airway cells, based on my postdoc work. This physiological self-organization emerges from the interplay between tissue geometry, developmental signaling, and tissue mechanics. (FIG. 17C)

In sum, this UV-based micropatterning platform provides a powerful means to standardize mini-organ generation and to dissect the fundamental principles of tissue self-organization with in vivo relevance. (FIG. 17C)

We are interested in developing organoid models of trachea-esophageal development. The trachea and esophagus arise from a shared embryonic foregut tube, which undergoes coordinated patterning and morphogenesis to separate into distinct respiratory and digestive structures. (FIG. 17D)

Lung specification marks the start of this process, followed by budding and partitioning events that ultimately produce the trachea and esophagus as separate, functional tubes. (FIG. 17D) When this process goes awry, it leads to congenital malformations in approximately 1 in every 3,000 U.S. births. Despite the severity of these defects, including esophageal atresia and tracheoesophageal fistulas, nearly 90% of cases have no known cause and no therapeutic options beyond surgical repair. (FIG. 17D)

We are leveraging stem cell-derived organoid models and bioengineered patterning systems to study how human trachea-esophageal morphogenesis unfolds in vitro. By recapitulating early foregut development under defined, reproducible conditions, we aim to uncover the molecular and mechanical cues that drive proper tube separation-and how these can be disrupted in disease. (FIG. 17D)

To do that, we devised a 13-day lung protocol that takes hPSCs cultured on micropatterns by modulating a set of signals we identified being critical for lung self-organization on micropatterns. (FIG. 17E)

Using this platform, we first generated endoderm and anterior endoderm cells, which are the precursors of lung progenitors. Then at the end, we add differentiation signals that robustly induce the generation of progenitor cells expressing a master regulator of lung development. At this stage, we observed the formation of bud-like structures resembling fetal lung buds in morphology. (FIG. 17E)

To investigate how the trachea and esophagus emerge from the foregut, we've developed a human stem cell-based model that recapitulates key steps of trachea-esophageal development in a controlled, in vitro setting. (FIG. 17F)

We begin with human pluripotent stem cells and differentiate them to definitive endoderm using WNT and Activin A. These cells are then seeded onto rectangular micropatterns to mimic the natural geometry of the foregut, which help control geometry and enhance reproducibility. After three days of patterning and treatment with BMP and TGF-beta inhibitors, we obtain patterned foregut tissues. (FIG. 17F)

At this stage, we can direct the tissue toward tracheal or esophageal fates using different morphogen combinations. Tracheal fate is induced with BMP, KGF, RA, and WNT, while esophageal identity is promoted using Noggin and EGF. (FIG. 17F)

This fate decision is reflected in marker expression: trachea-directed cultures robustly express NKX2.1, a lung and trachea marker, and suppress SOX2, whereas esophageal-directed cultures show the opposite pattern—with strong SOX2 and little to no NKX2.1. (FIG. 17F) This is further validated in our quantification, showing clear and reproducible segregation of cell fates, consistent with trachea and esophagus identity. (FIG. 17F)

Altogether, this platform gives us a powerful system to model human trachea-esophageal development in vitro and opens the door to dissecting the signaling and spatial cues that govern tube separation and lineage commitment—processes that are otherwise difficult to access in the human embryo. (FIG. 17F)

As we've shown, our in vitro system allows for precise fate specification of tracheal and esophageal lineages. But what we were particularly excited to observe was that these fates drive distinct morphogenetic behaviors—closely mirroring what we see during early embryo development. (FIG. 17G)

In vivo, the foregut undergoes regional patterning where lung buds emerge from the ventral side, eventually separating into the trachea and esophagus. These tissues adopt distinct geometries—tracheal domains are more bud-like, while esophageal structures elongate into tubes. (FIG. 17G)

When we pattern hPSC-derived foregut cells in vitro and specify them toward tracheal fate, we see a striking bud-like morphology emerge, as shown here by NKX2.1 expression and actin cytoskeleton organization. In side view, you can see a clear epithelial invagination forming over just 3 days. (FIG. 17G)

In contrast, esophageal-directed tissues—marked by SOX2—undergo tube-like elongation. From surface to side view, you can appreciate a continuous, tubular structure forming, again driven purely by fate specification and self-organization. (FIG. 17G)

What we're seeing is that fate isn't just a molecular identity—it's also instructive for morphogenesis. Tracheal and esophageal identities intrinsically guide tissue architecture, even in the absence of external mechanical cues. (FIG. 17G)

This gives us a unique opportunity to dissect how fate, signaling, and geometry interact to build organ-specific structure—something that's extremely difficult to study directly in the human embryo. (FIG. 17G)

To dissect how distinct morphogenetic programs emerge during trachea-esophageal development, we've implemented high-resolution live imaging of human foregut organoids. (FIG. 17H)

As we've shown, foregut endoderm can be directed toward bud-like tracheal morphogenesis or tube-like esophageal morphogenesis. But how these morphologies actually emerge from cellular behavior remains unclear. (FIG. 17H)

To capture this in real time, we engineered hPSC lines expressing fluorescent markers for nuclei (H2B), actin filaments (LifeAct), and tight junctions (ZO1). This allows us to monitor cell behavior, cytoskeletal remodeling, and epithelial organization as tissues undergo fate-directed morphogenesis. (FIG. 17H)

This platform is now allowing us to track how changes in cell identity translate into coordinated tissue-level shape changes-helping us answer a central question in organogenesis: How are cell fate and tissue morphogenesis linked across scales? (FIG. 17H)

Using live imaging of tight junctions with ZO1, we observed early compaction of cells prior to visible bud formation. This was especially pronounced in SOX2-positive domains, where we saw progressive crowding and organization of cells. (FIG. 17I) Zooming in, we confirmed this compaction at the single-cell level, alongside coordinated nuclear positioning, indicating active epithelial remodeling. Further analysis revealed that compacted cells preferentially express SOX2, an airway marker, and are surrounded by an actin-rich cortex—consistent with contractile activity. (FIG. 17I)

To test whether cell contractility is required for bud morphogenesis, we treated developing organoids with actin polymerization inhibitors-Cytochalasin D and Latrunculin B. These treatments disrupted epithelial structure, as seen in the F-actin images here, and prevented the emergence of bud-like morphologies. (FIG. 17I)

Interestingly, disrupting contractility not only impaired morphogenesis—it also skewed lineage proportions. Both inhibitors significantly reduced the number of SOX2+ cells, while SOX9+ cells expanded, indicating a link between mechanical cues and fate specification. (FIG. 17I)

Altogether, these results support a model in which cell contractility and actin dynamics are essential for initiating bud morphogenesis and for properly patterning tracheal identity. (FIG. 17I)

We have been investigating how tissue-level organization emerges during the earliest stages of bud morphogenesis. What we uncovered is that tissue behavior is not random—in fact, it exhibits strikingly synchronized patterns of self-organization. (FIG. 17J)

Using high-resolution live imaging of actin filaments and nuclei, we observed rotational patterns in the epithelial sheet—indicating that cells are not only behaving individually but are also coordinating with their neighbors to generate large-scale tissue dynamics. (FIG. 17J)

To quantify this, we performed orientation vector field analysis on ZO1-GFP-labeled tissues. This allowed us to measure the direction of cell alignment and the degree of local coherence—how consistently neighboring cells orient relative to one another. (FIG. 17J)

What we found was a striking circular pattern of tissue anisotropy—cells at the periphery align and rotate in a coordinated manner, forming a radial gradient in orientation coherence. (FIG. 17J)

This is reflected quantitatively here. Coherency increases with distance from the center, forming a radial pattern of anisotropy. However, when we inhibit actin polymerization with Cytochalasin D, this pattern collapses, indicating that actomyosin-based contractility is required for maintaining this synchronized tissue behavior. (FIG. 17J)

These results indicate that organ bud morphogenesis is not just a matter of local shape change—it involves emergent, coordinated behaviors across the entire tissue, driven by cytoskeletal dynamics. This raises exciting new questions about how mechanical and molecular cues integrate to guide organ-specific architecture. (FIG. 17J)

To scale our analysis of trachea-esophageal development, we've built an AI-enabled platform that combines high-content imaging with neural network-based phenotyping. (FIG. 17K)

We begin with large-scale generation of micropatterned human mini-organs in multi-well plates. In previous work, we deployed this platform to a high-throughput antiviral drug screen, profiling over 3,000 compounds. The assay performed well, highlighting its utility for phenotypic screening in engineered human tissues. (FIG. 17K) To profile trachea-esophageal development, each colony is stained for tissue identity markers—NKX2.1 and SOX2—as well as for F-actin to capture morphogenesis. This yields thousands of annotated images capturing both molecular identity and tissue architecture. (FIG. 17K)

To analyze these data, we use an unsupervised autoencoder neural network. This compresses each image into a set of latent features-a compact numerical representation that preserves biological variation without requiring manual labels. (FIG. 17K)

We then use UMAP to project these latent features into 2D space, revealing clear phenotypic clusters that correspond to BMP and Noggin dose responses. Remarkably, the model captures a continuous spectrum from trachea-like to esophagus-like states. (FIG. 17K)

To make this actionable, we then trained a classifier to assign differentiation scores to new samples. Here, you can see that low Noggin favors esophageal identity, high BMP induces tracheal fate, and control conditions fall in between in a precursor-like state. Using this classifier, we can now rapidly predict the differentiation outcome of each well in a screen—categorizing organoids as trachea, esophagus, or intermediate with high accuracy. (FIG. 17K)

Altogether, this approach allows for us to move beyond qualitative assessment of organoid identity, and toward scalable, quantitative, and predictive models of human TE development—opening the door to drug discovery, disease modeling, and systems-level analysis. (FIG. 17K)

To connect our in vitro model with clinical relevance, we asked whether our platform can help dissect patient-associated defects in tracheoesophageal development. (FIG. 17L)

We focused on Noggin, a gene associated with malformations of the trachea-esophageal tract. Analysis of fetal tissue from control and affected patients revealed consistent misexpression of Noggin in cases of esophageal atresia and tracheoesophageal fistula, indicating that disrupted BMP signaling can underline these congenital anomalies. (FIG. 17L)

To model this, we generated Noggin knockout hPSCs and differentiated them in our foregut patterning system. Compared to wild-type, Noggin-null tissues failed to properly differentiate into esophageal tissues, and instead demonstrated a bias towards a tracheal identity, mimicking the mixed or ambiguous identity observed in patient tissues. (FIG. 17L)

We next turned to our AI-based phenotyping platform to assess these differences in an unbiased way. Using UMAP to reduce the dimensionality of latent image features, we saw a clear separation between wild-type and Noggin KO tissues—even without any predefined labels. (FIG. 17L)

And when we applied our classifier to quantify differentiation scores, we found that Noggin knockout tissues showed dose-dependent failure to adopt a clear esophageal fate, especially under high Noggin. This result is consistent with a model where endogenous Noggin is required to restrict BMP signaling and preserve esophageal identity. (FIG. 17L)

Together, this work shows how we can use patient data, stem cell models, and AI-based phenotyping to reconstitute and predict congenital defects—and begin to uncover the developmental logic that goes awry in tracheoesophageal malformations. (FIG. 17L)

Key takeaways (FIG. 17M):

### Reconstitution of Trachea-Esophageal Development

1. Robust induction of tracheal vs esophageal fate on microchips
2. Tissue patterning-directed morphogenesis of the trachea-esophageal tract
3. Self-organization of organotypic tissue morphogenesis

### Self-Organization of Tissue Scale Biomechanics

1. Tissue anisotropy drives bud morphogenesis
2. Actomyosin contractility drives bud patterning and morphogenesis

### Predictive Profiling of TE Development and Disease

1. AI-based phenotypic profiling of trachea-esophageal development
2. Predictive modeling of developmental defects in response to disease-associated genetic insults
3. Predictive profiling of tissue morphogenesis in time and space in response to developmental signals.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1. (canceled)

2. A method for producing trachea or esophagus organoids, the method comprising:
seeding definitive endoderm cells onto rectangular microfabricated culture substrates comprising elongated confinement geometries, including rectangular and elliptical shapes, that spatially confines the cells;
contacting the cells with a TGF inhibitor and a BMP inhibitor to obtain patterned foregut tissues;
contacting the patterned foregut tissues with a culture medium cocktail comprising:
KGF, BMP4, retinoic acid and a WNT agonist for tracheal tissue determination or
KGF, Noggin, retinoic acid and a WNT agonist for esophageal tissue determination; and
culturing the definitive endoderm, foregut and patterned foregut cells in complete serum-free medium to obtain tracheal or esophageal organoids.

3. The method of claim 2, wherein the microfabricated culture substrates are a microchip.

4. The method of claim 3, wherein the microchip comprises rectangular hydrogel-coated domains (100-300 μm width; ≥500 μm length) separated by parallel hydrogel confinement walls to form well-like physical constraints.

5. The method of claim 4, wherein the microchip comprises:
micropatterned hydrogel substrates fabricated by projection sterolithography-mediated photopolymerization of poly(ethylene glycol) diacrylate (PEGDA), Lithium phenyl-2,4,6-trimethylbenzoylphosphinate (LAP) as photo-initiator and tartrazine as photo-absorber to generate hydrogel microstructures on glass-bottom culture vessels; and
the parallel hydrogel confinement walls with thickness exceeding cell height.

6. The method of claim 5, wherein the photopolymerization uses about 20% 6K-8 KDa poly(ethylene glycol) diacrylate (PEGDA), about 34 mM Lithium phenyl-2,4,6-trimethylbenzoylphosphinate (LAP) and about 2.25 mM tartrazine.

7. The method of claim 4, wherein the hydrogel is coated with laminin.

8. The method of claim 2, wherein the definitive endoderm cells are derived from embryonic stem cells (ESCs) or induced pluripotent stem cells (iPSCs).

9. The method of claim 8, wherein the stem cells are differentiated to the definitive endoderm cells using RPMI 1640 medium supplemented with 3 μM CHIR99021 and 100 ng/mL Activin A on day 1, 100 ng/ml Activin A and 0.2% deactivated fetal bovine serum (dFBS) on day 2, and 100 ng/ml Activin A plus 2% dFBS on day 3.

10. The method of claim 2, wherein the TGFβ inhibitor is SB431542 and the BMP inhibitor is Dorsomorphin.

11. The method of claim 10, wherein concentration of SB431542 is about 10 um and of Dorsomorphin is about 2 μm.

12. The method of claim 2, wherein the definitive endoderm cells are contacted with the TGFβ inhibitor and the BMP inhibitor for at least 3 days.

13. The method of claim 2, wherein the WNT agonist is CHIR9902.

14. The method of claim 13, wherein:
for the tracheal tissue determination, the culture medium cocktail provides a final concentration of the KGF of about 50 ng/ml, of the BMP4 of about 10 ng/ml, of the retinoic acid of about 100 nm and of the CHIR9902 of about 3 μM; and
for the esophageal tissue determination, the culture medium cocktail provides a final concentration of the KGF of about 50 ng/mL, of the Noggin of between about 10-200 ng/mL, of the retinoic acid of about 100 nm and of the CHIR9902 of about 3 μM.

15. The method of claim 13, wherein the patterned foregut cells are contacted with the culture medium cocktail for about 7 days.

16. The method of claim 13, wherein medium containing the cocktail is applied apically to cover the patterned foregut tissues, with daily medium changes.

17. The method of claim 2, wherein the complete serum-free medium comprises a complete serum-free differentiation medium (SFDM).

18. The method of claim 17, wherein the SFDM comprises Advanced DMEM/F 12, 15 mM HEPES, 2mM L-glutamine, 1×B27 supplement and 1×N2 supplement.

19. The method of claim 2, wherein the definitive endoderm, foregut and patterned foregut cells are cultured on laminin-coated micropattern glass coverslips.

20. The method of claim 2, wherein:
contacting the foregut cells with the culture medium cocktail for tracheal tissue determination results in formation of bud-like structures and expression of NKX2.1; and
culturing the foregut cells with the culture medium cocktail for esophageal tissue determination results in formation of elongated tubular epithelia and expression of SOX2.

21. The method of claim 2, wherein:
contacting the foregut cells with the tracheal differentiation cocktail promotes bud morphogenesis, characterized by increased lumen height and vertical aspect ratio, defined as a higher height relative to the longitudinal axis of the organoid; and
contacting the foregut cells with the esophageal differentiation cocktail induces elongated tubular morphogenesis, resulting in an increased longitudinal elongation of lumens and a higher longitudinal aspect ratio, reflecting greater length relative to width with continuous, single lumens across each micropattern.

22. The method of claim 2, wherein the definitive endoderm cells have a genetic mutation in, or abnormal expression of, a gene related to development of tracheal or esophageal tissue.

23. The method of claim 22, wherein the gene is CHD7, EFTUD2, ERCC4, FANCA, FANCB, FANCC, FAND1, FANG, FBN2, FGFR2, FLNA, FOXF1, FREM2, GDF3, GDF6, GLI3, MDM2, MEOX2, MID1, MKKS, MYCN, NKX2-1, NOG, PLEC1, PTEN, RARA, RARB, RBM8A, RIPK4, RMRP, SEMA3E, SMARCD1, SOX2, SOX4, SPECC1L, TBX1, TCOF1, WBP11, YY1, ZIC3, FOXF1, TBX1, FGFR2, BMP4, BMPR1A/B, GLI3, NOG, PTEN, or a combination thereof.

24. The microchip of claim 4.

25. A human tracheal or esophageal tissue or organoid derived from the method of claim 2.

26. A cultured tracheal or esophageal organoid derived from stem cells wherein:
the tracheal organoids exhibit radial tissue anisotropy surrounding bud primordia, including an edge-enriched orientation bias (increased Edge-to-Center Ratio); and
the esophageal organoids lack this feature.
